(19) United States
(12) Patent Application Publication
(10) Pub. No.: US 2024/0010637 A1
(43) Pub. Date: Jan. 11, 2024

(54) PROCESS FOR THE PREPARATION OF PYRIDAZINONE DERIVATIVES

(71) Applicant: SYNGENTA CROP PROTECTION AG, Basel (CH)

(72) Inventors: Raphael DUMEUNIER, Stein (CH); Tomas SMEJKAL, Stein (CH); Renaud BEAUDEGNIES, Stein (CH)

(73) Assignee: SYNGENTA CROP PROTECTION AG, Basel (CH)

(21) Appl. No.: 18/041,593

(22) PCT Filed: Aug. 13, 2021
(86) PCT No.: PCT/EP2021/072566

§ 371 Date: Feb. 14, 2023

(30) Foreign Application Priority Data
Aug. 14, 2020 (EP) 20191205.2
Jan. 15, 2021 (EP) 21151742.0

(51) Int. Cl. C07D 405/04; C07D 247/02

(52) U.S. Cl. CPC C07D 405/04; C07D 247/02

(57) ABSTRACT

The present invention provides, inter alia, a process for producing a compound of formula (I) wherein the substituents are as defined in claim 1. The present invention further provides intermediate compounds utilised in said process, and methods for producing said intermediate compounds.

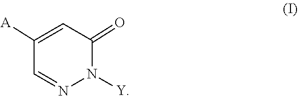

The present invention relates to a novel process for the synthesis of certain pyridazinone compounds. Such compounds are useful as intermediates in the synthesis of herbicidal pyridazine compounds, for example, those described in WO 2019/034757. Such compounds are typically produced via an alkylation of a pyridazine intermediate.

The alkylation of pyridazine intermediates is known (see for example WO 2019/034757), however, such a process has a number of drawbacks. Firstly, this approach often leads to a non-selective alkylation on either pyridazine nitrogen atom and secondly, an additional complex purification step is required to obtain the desired product. Thus, such an approach is not ideal for large scale production and therefore a new, more efficient synthesis method involving a selective alkylation is desired to avoid the generation of undesirable by-products.

Surprisingly, we have now found that the need for such a non-selective alkylation can be avoided by the use of certain pyridazinone intermediates which in turn can be converted to the desired herbicidal pyridazine compounds. Such a process is more convergent, which may be more cost effective and may produce less waste products.

Thus, according to the present invention there is provided a process for the preparation of a compound of formula (I):

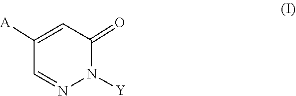

wherein
A is a 6-membered heteroaryl selected from the group consisting of formula A-I to A-VII below

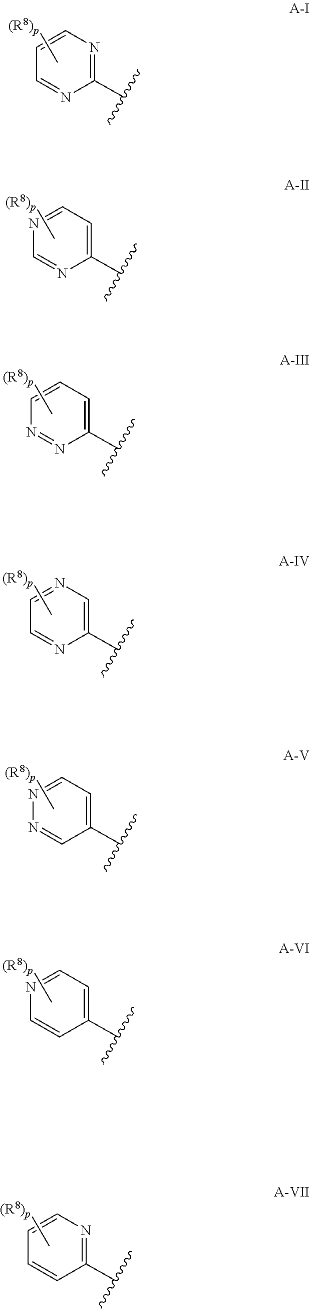

wherein the jagged line defines the point of attachment to the remaining part of a compound of formula (I), p is 0, 1 or 2; and
Y is hydrogen or the group Y-I below

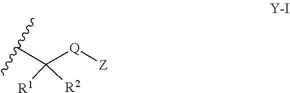

wherein the jagged line defines the point of attachment to the remaining part of a compound of formula (I); and
R¹ is hydrogen or methyl;
R² is hydrogen or methyl;
Q is (CR^{1a}R^{2b})ₘ;
m is 0, 1 or 2;
each R^{1a} and R^{2b} are independently selected from the group consisting of hydrogen, methyl, —OH and —NH₂;
Z is selected from the group consisting of —CN, —CH₂OR³, —CH(OR⁴)(OR⁴a), —C(OR⁴)(OR⁴a)(OR^{4b}), —C(O)OR¹⁰, —C(O)NR⁶R⁷ and —S(O)₂OR¹⁰; or
Z is selected from the group consisting of a group of formula Zₐ, Z_{b}, Z_{c}, Z_{d}, Zₑ and Z_{f} below

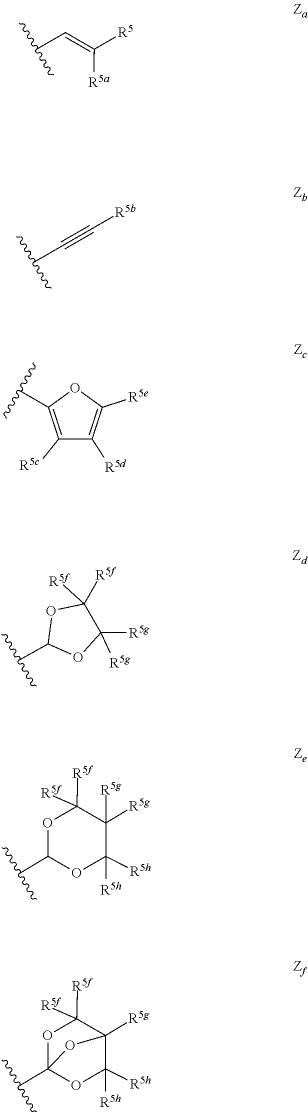

wherein the jagged line defines the point of attachment to the remaining part of a compound of formula (I); and
R³ is selected from the group consisting of hydrogen, —C(O)OR^{10a} and —C(O)R^{10a};
each R⁴, R^{4a} and R^{4b} are independently selected from hydrogen and C₁-C₆alkyl;
each R⁵, R^{5a}, R^{5b}, R^{5c}, R^{5d}, R^{5e}, R^{5f}, R^{5g} and R^{5h} are independently selected from the group consisting of hydrogen and C₁-C₆alkyl;
each R⁶ and R⁷ are independently selected from the group consisting of hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl; and
R^{10a} is selected from the group consisting of hydrogen, C₁-C₆alkyl, phenyl and benzyl;
said process comprising:
reacting a compound of formula (II):

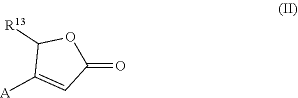

wherein A is as defined above;
R¹³ is selected from the group consisting of halogen, ═O, —OR¹⁶ and —NR¹⁴R¹⁵;
R¹⁴ and R¹⁵ are independently selected from the group consisting of hydrogen and C₁-C₆alkyl; or
R¹⁴ and R¹⁵ together with the nitrogen atom to which they are attached form a 4- to 6-membered heterocyclyl ring which optionally comprises one additional heteroatom individually selected from nitrogen, oxygen and sulfur; and
R¹⁶ is selected from the group consisting of hydrogen, C₁-C₆alkyl, —C(O)OR^{10a} and —C(O)R^{10a};
R^{10a} is as defined herein;
with a compound of formula (III):

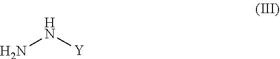

wherein Y is as defined herein, to produce a compound of formula (I);

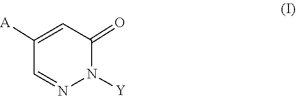

wherein A and Y are as defined herein.

According to a second aspect of the invention, there is provided an intermediate compound of formula (II)

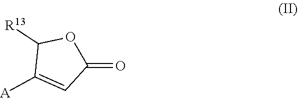

wherein A and R¹³ are as defined herein.

According to a third aspect of the invention, there is further provided an intermediate compound of formula (IV)

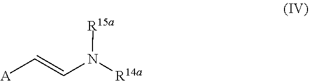

wherein A, R^{14a} and R^{15a} are as defined herein.

According to a fourth aspect of the invention, there is provided the use of a compound of formula (IV) for preparing a compound of formula (I).

According to a fifth aspect of the invention, there is provided the use of a compound of formula (VI) for preparing a compound of formula (I)

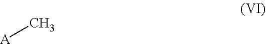

wherein A is as defined herein.

According to a sixth aspect of the invention, there is provided the use of a compound of formula (III) for preparing a compound of formula (I)

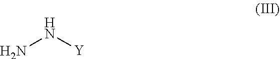

wherein Y is as defined herein.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl, ethyl, n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. Examples of C₁-C₆alkoxy include, but are not limited to, methoxy, ethoxy, propoxy, iso-propoxy and t-butoxy.

Compounds of formula (I) wherein Y is group Y-I below

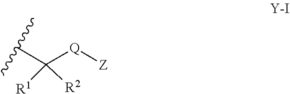

and m is 0 may be represented by a compound of formula (I-Ia) as shown below:

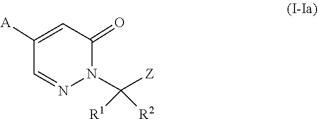

wherein R¹, R², A and Z are as defined for compounds of formula (I).

Compounds of formula (I) wherein Y is Y-I and m is 1 may be represented by a compound of formula (I-Ib) as shown below:

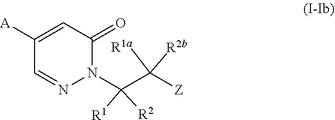

wherein R¹, R², R^{1a}, R^{2b}, A and Z are as defined for compounds of formula (I).

Compounds of formula (I) wherein Y is Y-I and m is 2 may be represented by a compound of formula (I-Ic) as shown below:

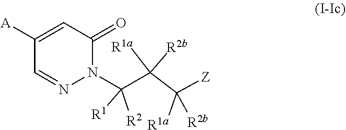

wherein R¹, R², R^{1a}, R^{2b}, A and Z are as defined for compounds of formula (I).

Compounds of formula (III) wherein Y is group Y-I below

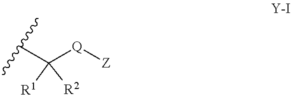

and m is 0 may be represented by a compound of formula (III-a) as shown below:

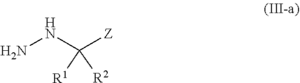

wherein R¹, R², A and Z are as defined herein.

Compounds of formula (III) wherein Y is Y-I and m is 1 may be represented by a compound of formula (III-b) as shown below:

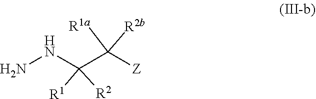

wherein R¹, R², R^{1a}, R^{2b}, A and Z are as defined herein.

Compounds of formula (III) wherein Y is Y-I and m is 2 may be represented by a compound of formula (III-c) as shown below:

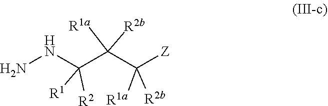

wherein R¹, R², R^{1a}, R^{2b}, A and Z are as defined herein.

The following list provides definitions, including preferred definitions, for substituents m, p, A, Q, Y, Z, Z², R¹, R², R^{1a}, R^{2b}, R³, R⁴, R^{4a}, R^{4b}, R⁵, R^{5a}, R^{5b}, R^{5c}, R^{5d}, R^{5e}, R^{5f}, R^{5g}, R^{5h}, R⁶, R⁷, R⁸, R¹⁰, R^{10a}, R¹³, R^{13a}, R^{13b}, R¹⁴, R¹⁵, R^{14a}, R^{15a}, R¹⁶, R²², R²³, R²⁴, R²⁵, R²⁶ with reference to the process according to the invention. For any one of these substituents, any of the definitions given below may be combined with any definition of any other substituent given below or elsewhere in this document.

A is a 6-membered heteroaryl selected from the group consisting of formula A-I to A-VII below

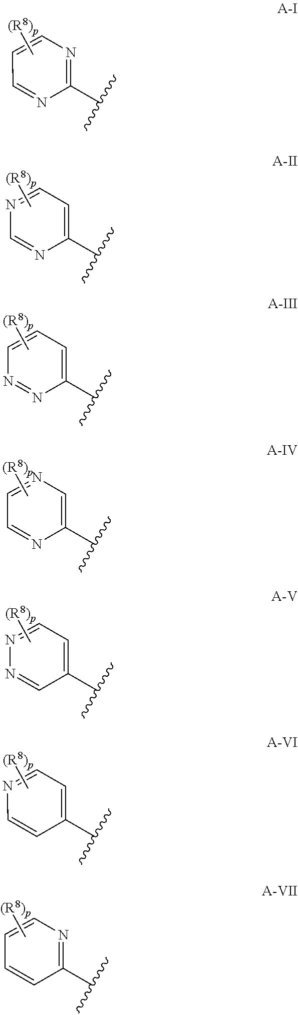

wherein the jagged line defines the point of attachment to the remaining part of a compound of formula (I), p is 0, 1 or 2 (preferably, p is 0 or 1, more preferably, p is 0).

Preferably, A is a 6-membered heteroaryl selected from the group consisting of formula A-I, A-II, A-III, A-IV, A-V and A-VII below

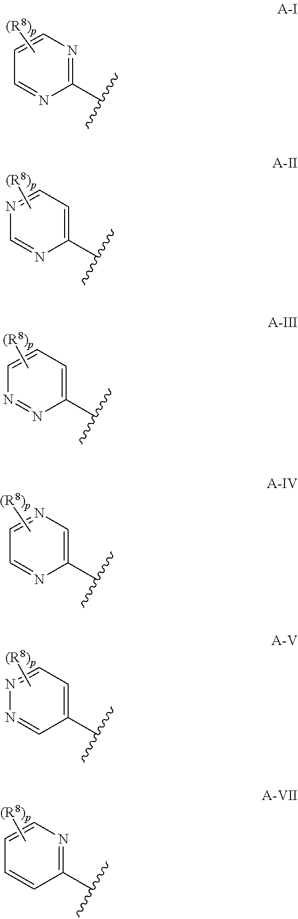

wherein the jagged line defines the point of attachment to the remaining part of a compound of formula (I), p is 0, 1 or 2 (preferably, p is 0 or 1, more preferably, p is 0).

More preferably, A is a 6-membered heteroaryl selected from the group consisting of formula A-Ia, A-IIa, A-IIIa, A-IVa, A-Va and A-VIIa below

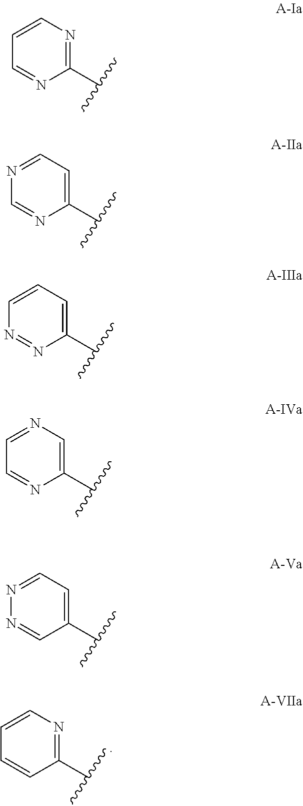 [SMILES: *c1ncccn1 ; *c1ccncn1 ; *c1cccnn1 ; *c1cnccn1 ; *c1ccnnc1 ; *c1ccccn1]

wherein the jagged line defines the point of attachment to the remaining part of a compound of formula (I).

Even more preferably, A is selected from the group consisting of formula A-Ia to A-IIIa below,

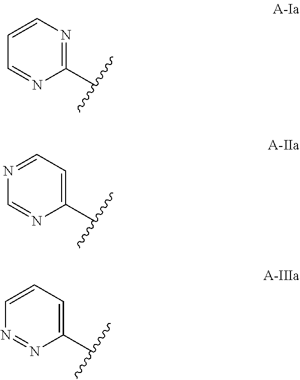 [SMILES: *c1ncccn1 ; *c1ccncn1 ; *c1cccnn1]

wherein the jagged line defines the point of attachment to the remaining part of a compound of formula (I).

Most preferably, A is the group A-Ia or A-IIIa.

Y is hydrogen or the group Y-I below

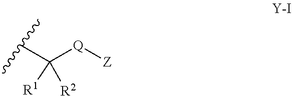

In one embodiment, Y is hydrogen.

In another embodiment, Y is the group Y-I.

R¹ is hydrogen or methyl, preferably R¹ is hydrogen.

R² is hydrogen or methyl, preferably R² is hydrogen.

In a preferred embodiment R¹ and R² are hydrogen.

Q is (CR^{1a}R^{2b})ₘ. Preferably, Q is CH₂.

m is 0, 1 or 2, preferably m is 1 or 2. Most preferably, m is 1.

each R^{1a} and R^{2b} are independently selected from the group consisting of hydrogen, methyl, —OH and —NH₂. More preferably, each R^{1a} and R^{2b} are independently selected from the group consisting of hydrogen and methyl. Most preferably R^{1a} and R^{2b} are hydrogen.

Z is selected from the group consisting of —CN, —CH₂OR³, —CH(OR⁴)(OR⁴a), —C(OR⁴)(OR⁴a)(OR^{4b}), —C(O)OR¹⁰, —C(O)NR⁶R⁷ and —S(O)₂OR¹⁰. Preferably, Z is selected from the group consisting of —CN, —CH₂OR³, —C(O)OR¹⁰, —C(O)NR⁶R⁷ and —S(O)₂OR¹⁰. More preferably, Z is selected from the group consisting of—CN, —CH₂OH, —C(O)OR¹⁰, —C(O)NH₂ and —S(O)₂OR¹⁰. Even more preferably, Z is selected from the group consisting of —CN, —CH₂OH, —C(O)OR¹⁰ and —S(O)₂OR¹⁰. Yet even more preferably still, Z is selected from the group consisting of —CN, —C(O)OR¹⁰ and —S(O)₂OR¹⁰. Yet even more preferably still, Z is selected from the group consisting of —CN, —C(O)OCH₂CH₃, —C(O)OC(CH₃)₃, —C(O)OH, —S(O)₂OCH₂C(CH₃)₃ and —S(O)₂OH. Yet further more preferably still, Z is selected from the group consisting of —CN, —C(O)OCH₂CH₃, —C(O)OC(CH₃)₃ and —C(O)OH.

In an alternative embodiment Z is selected from the group consisting of a group of formula Zₐ, Z_{b}, Z_{c}, Z_{d}, Zₑ and Z_{f} below

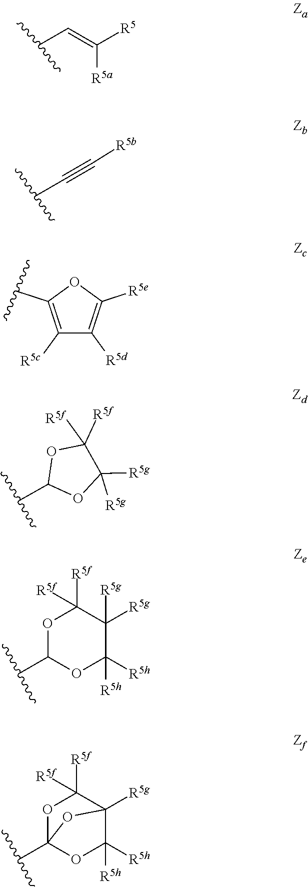

wherein the jagged line defines the point of attachment to the remaining part of a compound of formula (I). Preferably, Z is selected from the group consisting of a group of formula Zₐ, Z_{b}, Z_{d}, Zₑ and Z_{f}. More preferably, Z is selected from the group consisting of a group of formula Zₐ, Z_{d} and Zₑ.

In another embodiment of the invention Z is —C(O)OR¹⁰ and R¹⁰ is hydrogen or C₁-C₆alkyl. Preferably Z is —C(O)OCH₂CH₃.

In another embodiment of the invention Z is selected from the group consisting of —CN, —CH₂OH, —C(O)OR¹⁰ and —S(O)₂OR¹⁰, or Z is selected from the group consisting of a group of formula Zₐ, Z_{d} and Zₑ. Preferably, Z is selected from the group consisting of —CN, —CH₂OH, —C(O)OR¹⁰, —S(O)₂OR¹⁰ and —CH═CH₂.

The skilled person would appreciate that Z² below is a subset of Z for specific embodiments of the invention.

Z² is —C(O)OH or —S(O)₂OH. Preferably, Z² is —C(O)OH.

R³ is selected from the group consisting of hydrogen, —C(O)OR^{10a} and —C(O)R^{10a}. Preferably, R³ is hydrogen or —C(O)OR^{10a}. Most preferably, R³ is hydrogen.

Each R⁴, R^{4a} and R^{4b} are independently selected from C₁-C₆alkyl. Preferably, each R⁴, R^{4a} and R^{4b} are methyl.

Each R⁵, R^{5a}, R^{5b}, R^{5c}, R^{5d}, R^{5e}, R^{5f}, R^{5g} and R^{5h} are independently selected from the group consisting of hydrogen and C₁-C₆alkyl. More preferably, each R⁵, R^{5a}, R^{5b}, R^{5c}, R^{5d}, R^{5e}, R^{5f}, R^{5g} and R^{5h} are independently selected from the group consisting of hydrogen and methyl. Most preferably, each R⁵, R^{5a}, R^{5b}, R^{5c}, R^{5d}, R^{5e}, R^{5f}, R^{5g} and R^{5h} are hydrogen.

Each R⁶ and R⁷ are independently selected from the group consisting of hydrogen and C₁-C₆alkyl. Preferably, each R⁶ and R⁷ are independently hydrogen or methyl. Most preferably, each R⁶ and R⁷ are hydrogen.

Each R⁸ is independently selected from the group consisting of halo, —NH₂, methyl and methoxy. Preferably, R⁸ is halo (preferably, chloro or bromo) or methyl. More preferably, R⁸ is chloro or bromo.

R¹⁰ is selected from the group consisting of hydrogen, C₁-C₆alkyl, phenyl and benzyl. Preferably, R¹⁰ is the group consisting of hydrogen and C₁-C₆alkyl. More preferably, R¹⁰ is selected from the group consisting of hydrogen, methyl, ethyl, iso-propyl, 2,2-dimethylpropyl and tert-butyl.

R^{10a} is selected from the group consisting of hydrogen, C₁-C₆alkyl, phenyl and benzyl. Preferably, R^{10a} is selected from the group consisting of hydrogen, C₁-C₆alkyl and phenyl. More preferably, R^{10a} is the group consisting of hydrogen and C₁-C₆alkyl.

In one embodiment of the invention, R¹⁰ is ethyl or tert-butyl. Preferably, R¹⁰ is ethyl.

R¹³ is selected from the group consisting of halogen, ═O, —OR¹⁶ and —NR¹⁴R¹⁵. Preferably, R¹³ is selected from the group consisting of chloro, —OR¹⁶ and —NR¹⁴R¹⁵. More preferably, R¹³ is selected from the group consisting of chloro, —OH, —OMe, —OEt, —N(Me)₂, morpholinyl, piperidinyl and pyrrolidinyl. Even more preferably, R¹³ is selected from the group consisting of —OH, —N(Me)₂, morpholinyl, piperidinyl and pyrrolidinyl. Yet even more preferably still, R¹³ is selected from the group consisting of—OH, morpholinyl, piperidinyl and pyrrolidinyl. Yet even more preferably still, R¹³ is —OH or morpholinyl. Most preferably, R¹³ is morpholinyl.

Each R^{13a} and R^{13b} are independently selected from the group consisting of halogen, —OR¹⁶ and —NR¹⁴R¹⁵. Preferably, each R^{13a} and R^{13b} are independently selected from the group consisting of chloro, —OH, —OMe, —OEt, —N(Me)₂, morpholinyl, piperidinyl and pyrrolidinyl. More preferably, each R^{13a} and R^{13b} are independently selected from the group consisting of —OH, morpholinyl, piperidinyl and pyrrolidinyl.

Alternatively, R^{13a} and R^{13b} together are ═O.

R¹⁴ and R¹⁵ are independently selected from the group consisting of hydrogen and C₁-C₆alkyl.

Preferably, R¹⁴ and R¹⁵ are independently selected from the group consisting of hydrogen, methyl and ethyl. Even more preferably, R¹⁴ and R¹⁵ are independently hydrogen or methyl. Most preferably, R¹⁴ and R¹⁵ are methyl.

Alternatively, R¹⁴ and R¹⁵ together with the nitrogen atom to which they are attached form a 4- to 6-membered heterocyclyl ring which optionally comprises one additional heteroatom individually selected from nitrogen, oxygen and sulfur. Preferably, R¹⁴ and R¹⁵ together with the nitrogen atom to which they are attached form a 4- to 6-membered heterocyclyl ring which optionally comprises one additional heteroatom individually selected from nitrogen and oxygen. More preferably, R¹⁴ and R¹⁵ together with the nitrogen atom to which they are attached form a 5- to 6-membered heterocyclyl ring which optionally comprises one additional heteroatom individually selected from nitrogen and oxygen. Even more preferably, R¹⁴ and R¹⁵ together with the nitrogen atom to which they are attached form a 5- to 6-membered heterocyclyl ring which optionally comprises one additional oxygen atom. Most preferably, R¹⁴ and R¹⁵ together with the nitrogen atom to which they are attached form a morpholinyl, piperidinyl or pyrrolidinyl group.

R^{14a} and R^{15a} are independently selected from the group consisting of hydrogen, C₁-C₆alkyl, C₁-C₆haloalkyl and phenyl. Preferably, R^{14a} and R^{15a} are independently selected from the group consisting of hydrogen and C₁-C₆alkyl. More preferably, R^{14a} and R^{15a} are independently selected from the group consisting of hydrogen, methyl and ethyl. Even more preferably, R^{14a} and R^{15a} are independently hydrogen or methyl. Most preferably, R^{14a} and R^{15a} are methyl.

Alternatively, R^{14a} and R^{15a} together with the nitrogen atom to which they are attached form a 4- to 6-membered heterocyclyl ring which optionally comprises one additional heteroatom individually selected from nitrogen, oxygen and sulfur. Preferably, R^{14a} and R^{15a} together with the nitrogen atom to which they are attached form a 4- to 6-membered heterocyclyl ring which optionally comprises one additional heteroatom individually selected from nitrogen and oxygen. More preferably, R^{14a} and R^{15a} together with the nitrogen atom to which they are attached form a 5- to 6-membered heterocyclyl ring which optionally comprises one additional heteroatom individually selected from nitrogen and oxygen. Even more preferably, R^{14a} and R^{15a} together with the nitrogen atom to which they are attached form a 5- to 6-membered heterocyclyl ring which optionally comprises one additional oxygen atom. Even more preferably still, R^{14a} and R^{15a} together with the nitrogen atom to which they are attached form a morpholinyl, piperidinyl or pyrrolidinyl group. Most preferably, R^{14a} and R^{15a} together with the nitrogen atom to which they are attached form a pyrrolidinyl group.

R¹⁶ is selected from the group consisting of hydrogen, C₁-C₆alkyl, —C(O)OR^{10a} and —C(O)R^{10a}. Preferably, R¹⁶ is selected from the group consisting of hydrogen, C₁-C₆alkyl and —C(O)OR^{10a}. More preferably, R¹⁶ is selected from the group consisting of hydrogen and C₁-C₆alkyl. Even more preferably, R¹⁶ is selected from the group consisting of hydrogen, methyl and ethyl.

Preferably, the compound of formula (I) is further subjected to a sulfurization, alkylation (if necessary), oxidative desulfurization, hydrolysis, oxidation and/or a salt exchange (i.e converted) to give an agronomically acceptable salt of formula (Ia) or a zwitterion of formula (Ib),

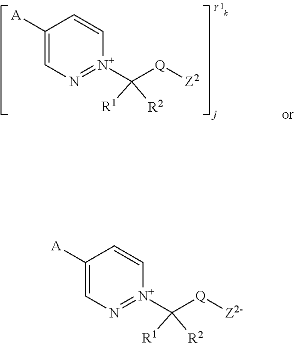

wherein Y¹ represents an agronomically acceptable anion and j and k represent integers that may be selected from 1, 2 or 3 (preferably, Y¹ is Cl⁻ and j and k are 1), and A, R¹, R² and Q are as defined herein and Z² is —C(O)OH or —S(O)₂OH (the skilled person would appreciate that Z²⁻ represents —C(O)O⁻ or —S(O)₂O⁻).

The present invention further provides an intermediate compound of formula (II):

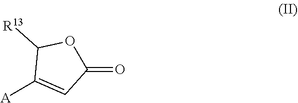

wherein A and R¹³ are as defined herein.

Preferably, in an intermediate compound of formula (II),

A is a 6-membered heteroaryl selected from the group consisting of formula A-Ia, A-IIa, and A-IIIa below

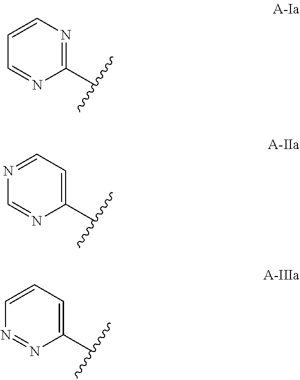 [SMILES: *c1ncccn1 ; *c1ccncn1 ; *c1cccnn1]

wherein the jagged line defines the point of attachment to the remaining part of a compound of formula (II) (preferably, A is the group A-Ia or A-IIIa); and
R¹³ is selected from the group consisting of chloro, —OH, —OMe, —OEt, —N(Me)₂, morpholinyl, piperidinyl and pyrrolidinyl.

More preferably, the intermediate compound of formula (II) is selected from the group consisting of a compound of formula (II-I), (II-II), (II-III), (II-IV), (II-V), (II-VI), (II-VII), (II-VIII), (II-IX), (II-X), (II-XI), (II-XII), (II-XIII), (II-XIV), (II-XV), (II-XVI), (II-XVII), (II-XVIII), (II-XIX), (II-XX), (II-XXI), (II-XXII), (II-XXIII) and (II-XXIV) below,

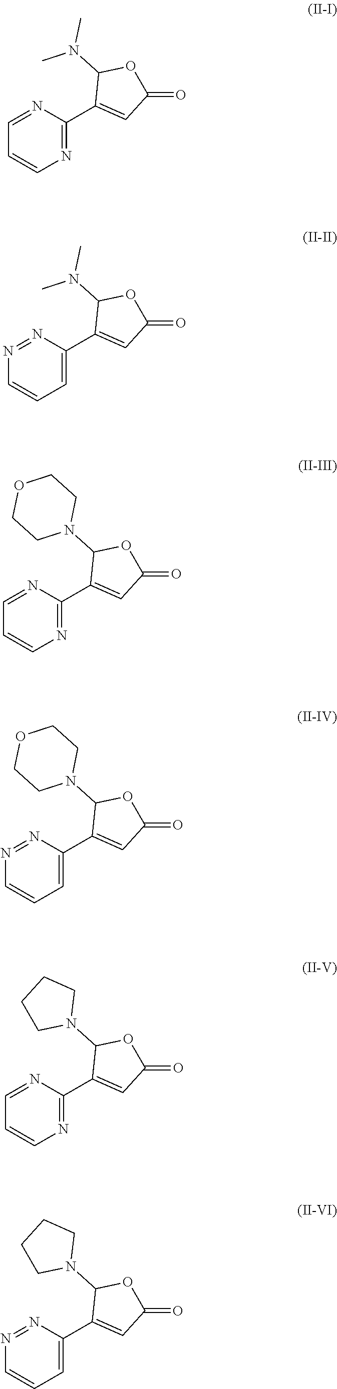 [SMILES: CN(C)C1OC(=O)C=C1c1ncccn1 ; CN(C)C1OC(=O)C=C1c1cccnn1 ; O=C1C=C(c2ncccn2)C(N2CCOCC2)O1 ; O=C1C=C(c2cccnn2)C(N2CCOCC2)O1 ; O=C1C=C(c2ncccn2)C(N2CCCC2)O1 ; O=C1C=C(c2cccnn2)C(N2CCCC2)O1]

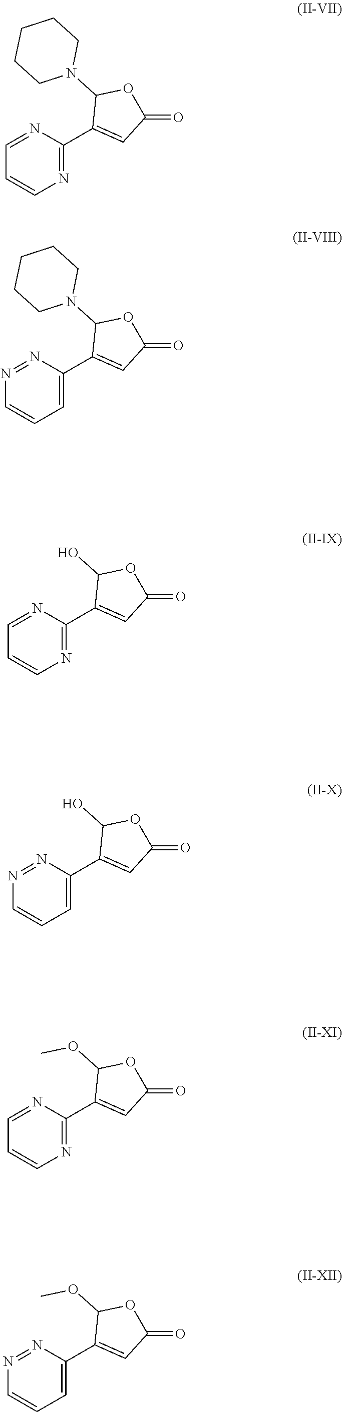 [SMILES: O=C1C=C(c2ncccn2)C(N2CCCCC2)O1 ; O=C1C=C(c2cccnn2)C(N2CCCCC2)O1 ; O=C1C=C(c2ncccn2)C(O)O1 ; O=C1C=C(c2cccnn2)C(O)O1 ; COC1OC(=O)C=C1c1ncccn1 ; COC1OC(=O)C=C1c1cccnn1]

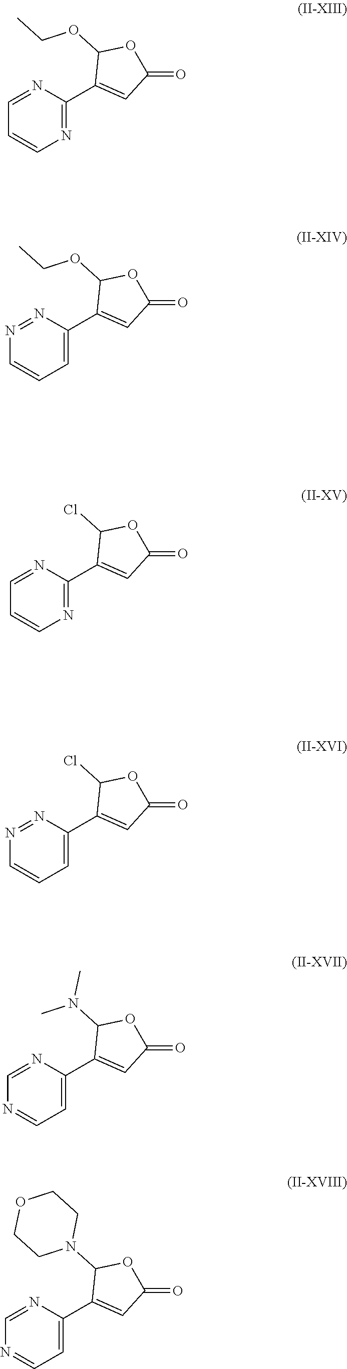 [SMILES: CCOC1OC(=O)C=C1c1ncccn1 ; CCOC1OC(=O)C=C1c1cccnn1 ; O=C1C=C(c2ncccn2)C(Cl)O1 ; O=C1C=C(c2cccnn2)C(Cl)O1 ; CN(C)C1OC(=O)C=C1c1ccncn1 ; O=C1C=C(c2ccncn2)C(N2CCOCC2)O1]

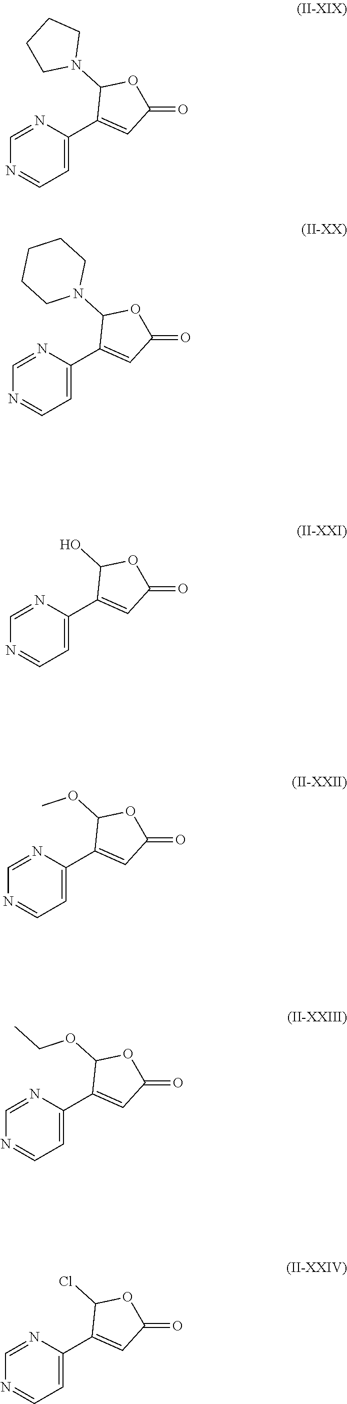 [SMILES: O=C1C=C(c2ccncn2)C(N2CCCC2)O1 ; O=C1C=C(c2ccncn2)C(N2CCCCC2)O1 ; O=C1C=C(c2ccncn2)C(O)O1 ; COC1OC(=O)C=C1c1ccncn1 ; CCOC1OC(=O)C=C1c1ccncn1 ; O=C1C=C(c2ccncn2)C(Cl)O1]

Even more preferably, the intermediate compound of formula (II) is selected from the group consisting of a compound of formula (II-I), (II-II), (II-III), (II-IV), (II-V), (II-I), (II-VII), (II-VIII), (II-IX), (II-X), (II-XI), (II-XII), (II-XIII), (II-XIV), (II-XV) and (II-XVI) below,

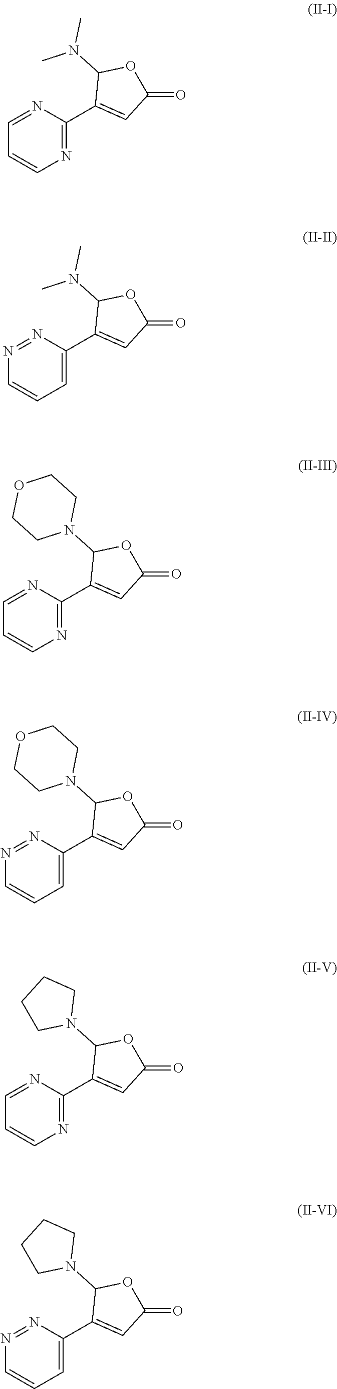 [SMILES: CN(C)C1OC(=O)C=C1c1ncccn1 ; CN(C)C1OC(=O)C=C1c1cccnn1 ; O=C1C=C(c2ncccn2)C(N2CCOCC2)O1 ; O=C1C=C(c2cccnn2)C(N2CCOCC2)O1 ; O=C1C=C(c2ncccn2)C(N2CCCC2)O1 ; O=C1C=C(c2cccnn2)C(N2CCCC2)O1]

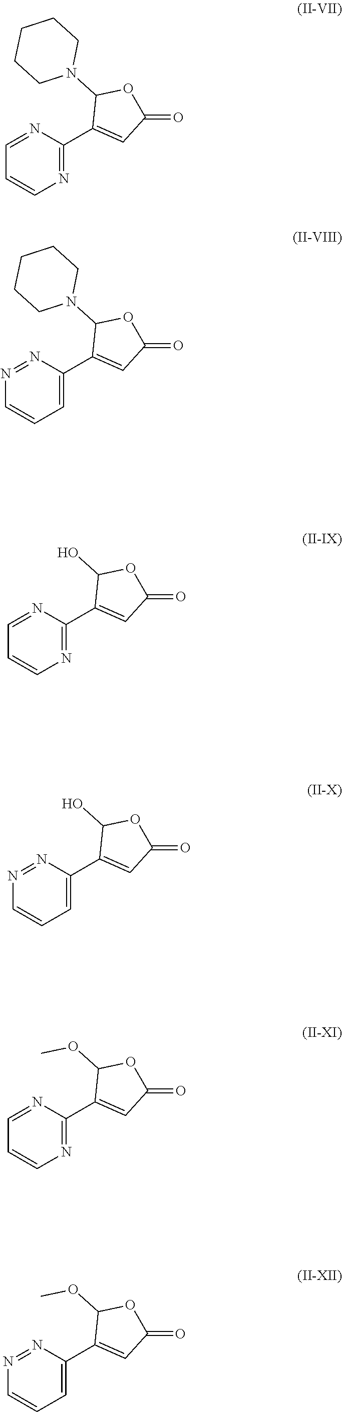 [SMILES: O=C1C=C(c2ncccn2)C(N2CCCCC2)O1 ; O=C1C=C(c2cccnn2)C(N2CCCCC2)O1 ; O=C1C=C(c2ncccn2)C(O)O1 ; O=C1C=C(c2cccnn2)C(O)O1 ; COC1OC(=O)C=C1c1ncccn1 ; COC1OC(=O)C=C1c1cccnn1]

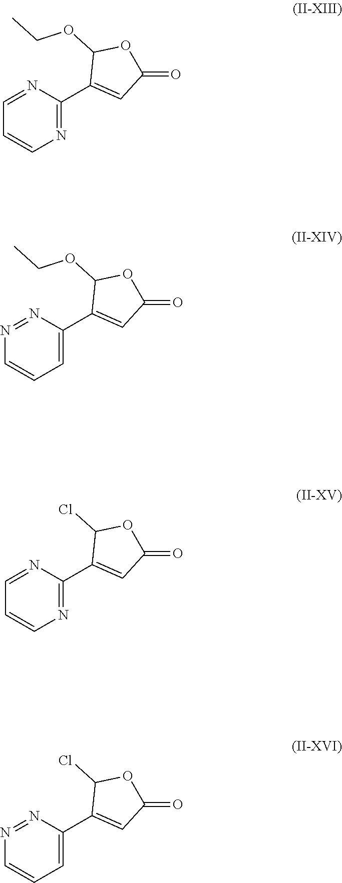

Even more preferably still, the intermediate compound of formula (II) is selected from the group consisting of a compound of formula (II-I), (II-Il), (II-III), (II-IV), (II-V), (II-VI), (II-VII), (II-VIII), (II-IX) and (II-X) below,

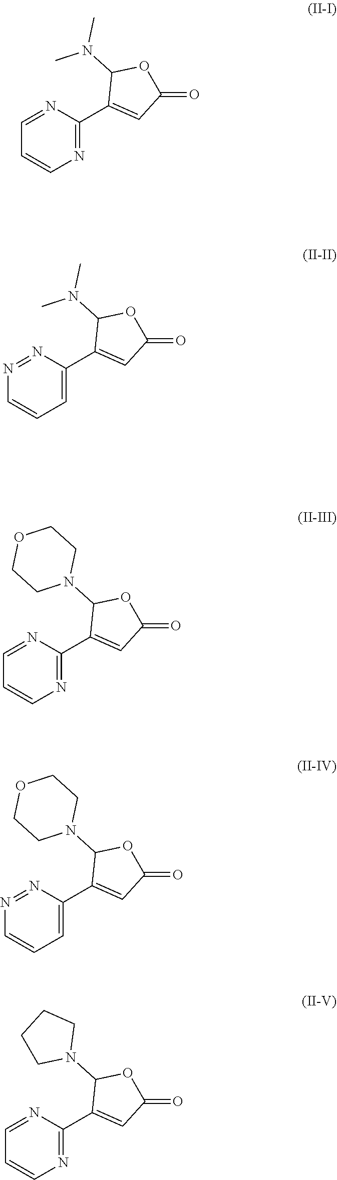 [SMILES: CN(C)C1OC(=O)C=C1c1ncccn1 ; CN(C)C1OC(=O)C=C1c1cccnn1 ; O=C1C=C(c2ncccn2)C(N2CCOCC2)O1 ; O=C1C=C(c2cccnn2)C(N2CCOCC2)O1 ; O=C1C=C(c2ncccn2)C(N2CCCC2)O1]

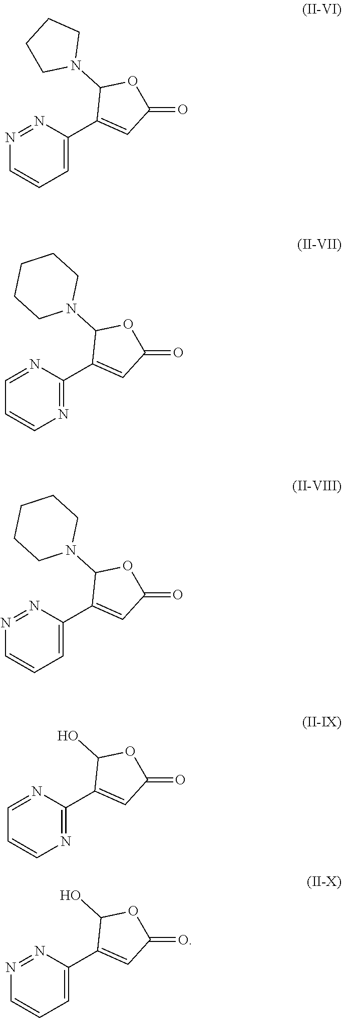 [SMILES: O=C1C=C(c2cccnn2)C(N2CCCC2)O1 ; O=C1C=C(c2ncccn2)C(N2CCCCC2)O1 ; O=C1C=C(c2cccnn2)C(N2CCCCC2)O1 ; O=C1C=C(c2ncccn2)C(O)O1 ; O=C1C=C(c2cccnn2)C(O)O1]

The present invention further provides an intermediate compound of formula (IV)

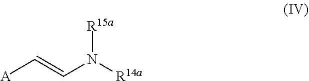

wherein A is a 6-membered heteroaryl selected from the group consisting of formula A-I, A-II, A-III, A-IV, A-V and A-VII below

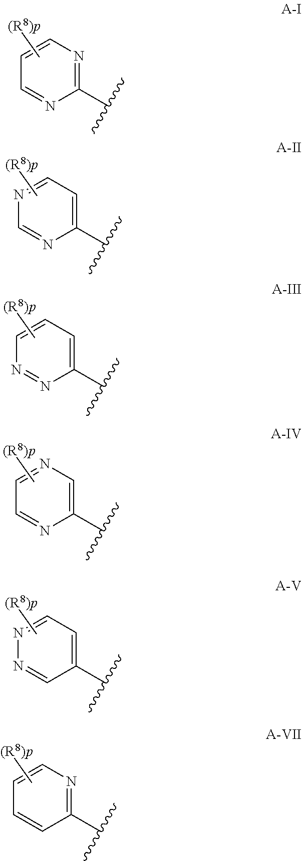

wherein the jagged line defines the point of attachment to the remaining part of a compound of formula (I), p and R⁸ are as defined; and
R^{14a} and R^{15a} are independently selected from the group consisting of C₂-C₆alkyl, C₁-C₆haloalkyl and phenyl; or
R^{14a} and R^{15a} together with the nitrogen atom to which they are attached form a 4- to 6-membered heterocyclyl ring which optionally comprises one additional heteroatom individually selected from nitrogen, oxygen and sulfur.

Preferably, in an intermediate compound of formula (IV),

A is a 6-membered heteroaryl selected from the group consisting of formula A-Ia, A-IIa, and A-IIIa below

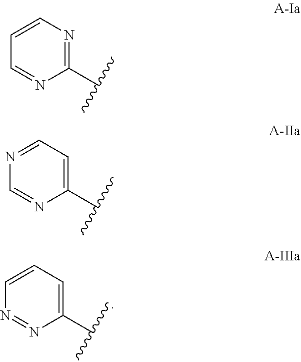 [SMILES: *c1ncccn1 ; *c1ccncn1 ; *c1cccnn1]

wherein the jagged line defines the point of attachment to the remaining part of a compound of formula (IV) (preferably, A is the group A-Ia or A-IIIa); and
R^{14a} and R^{15a} are independently selected from C₂-C₆alkyl; or
R^{14a} and R^{15a} together with the nitrogen atom to which they are attached form a 4- to 6-membered heterocyclyl ring which optionally comprises one additional oxygen atom (preferably, R^{14a} and R^{15a} together with the nitrogen atom to which they are attached form a morpholinyl, piperidinyl or pyrrolidinyl group).

More preferably, the compound of formula (IV) is selected from the group consisting of a compound of formula (IV-I), (IV-II), (IV-III), (IV-IV), (IV-V), (IV-VI), (IV-VII), (IV-VIII) and (IV-IX) below,

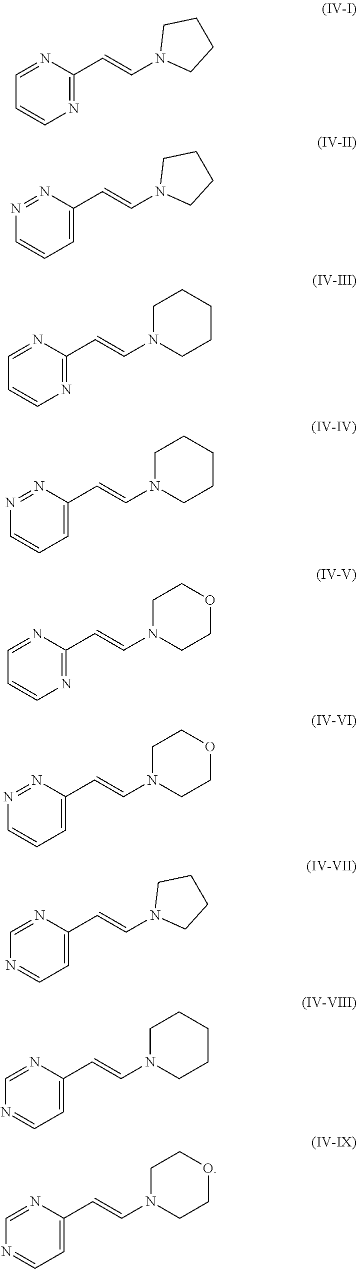

Even more preferably, the compound of formula (IV) is selected from the group consisting of a compound of formula (IV-I), (IV-II), (IV-III), (IV-IV), (IV-V) and (IV-VI) below,

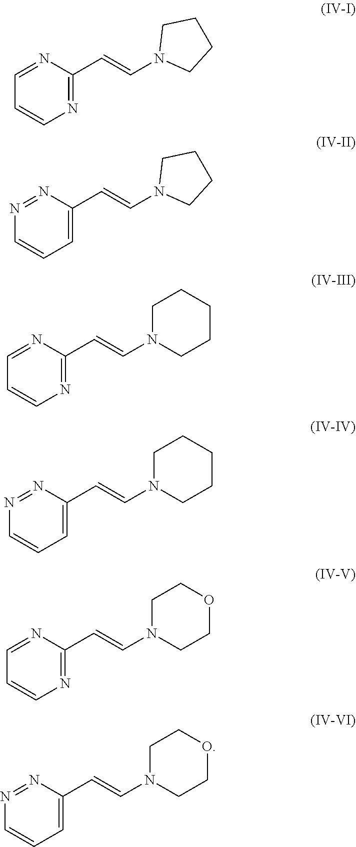 [SMILES: C(=C\N1CCCC1)/c1ncccn1 ; C(=C\N1CCCC1)/c1cccnn1 ; C(=C\N1CCCCC1)/c1ncccn1 ; C(=C\N1CCCCC1)/c1cccnn1 ; C(=C\N1CCOCC1)/c1ncccn1 ; C(=C\N1CCOCC1)/c1cccnn1]

In an alternative embodiment of the invention the compound of formula (IV) is a compound of formula (IV-Ia), (IV-IIa) or (IV-IIIa) below,

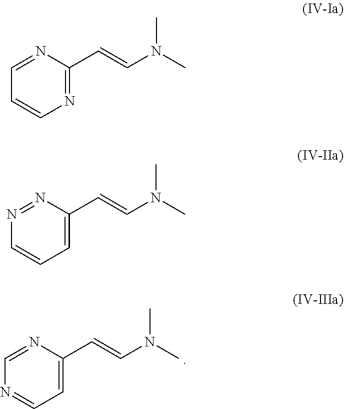

In one embodiment of the invention there is provided the use of a compound of formula (VI) for preparing a compound of formula (I)

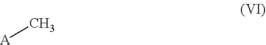

wherein A is as defined herein.

Preferably, there is provided the use of a compound of formula (VI) for preparing a compound of formula (I) wherein
A is selected from the group consisting of formula A-Ia to A-IIIa below,

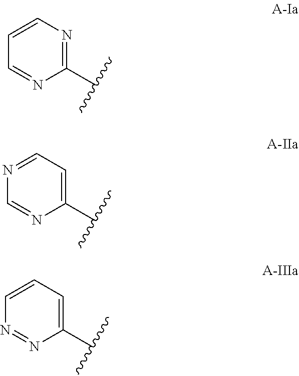 [SMILES: *c1ncccn1 ; *c1ccncn1 ; *c1cccnn1]

wherein the jagged line defines the point of attachment to the remaining part of a compound of formula (VI).

More preferably, there is provided the use of a compound of formula (VI-I), (VI-II) or a compound of formula (VI-III) below

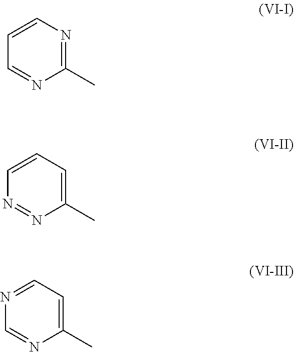

for preparing a compound of formula (I).

Even more preferably, there is provided the use of a compound of formula (VI-I)) or a compound of formula (VI-II) below

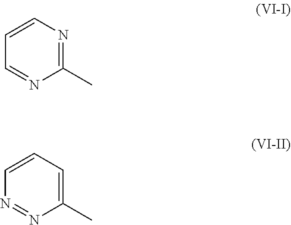

for preparing a compound of formula (I).

Compounds of formula (VI) are either known in the literature or may be prepared by known literature methods.

In another embodiment of the invention, there is provided the use of a compound of formula (III) for preparing a compound of formula (I)

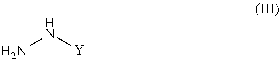

wherein Y is as defined herein.

Preferably, there is provided the use of a compound of formula (III) wherein
Y is hydrogen or the group Y-I below

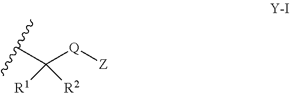

wherein the jagged line defines the point of attachment to the remaining part of a compound of formula (III); and
R¹ is hydrogen;
R² is hydrogen;
Q is (CR^{1a}R^{2b})ₘ;
m is 1;
each R^{1a} and R^{2b} are hydrogen;
Z is selected from the group consisting of —CN, —CH₂OH, —C(O)OR¹⁰, and —S(O)₂OR¹⁰ (preferably —CN, —C(O)OR¹⁰, and —S(O)₂OR¹⁰); and
R¹⁰ is selected from the group consisting of hydrogen and C₁-C₆alkyl (preferably, R¹⁰ is selected from the group consisting of hydrogen, methyl, ethyl, iso-propyl, 2,2-dimethylpropyl and tert-butyl).

More preferably, there is provided the use of a compound of formula (III-I), (III-II), (III-III), (III-IV) or (III-V) below

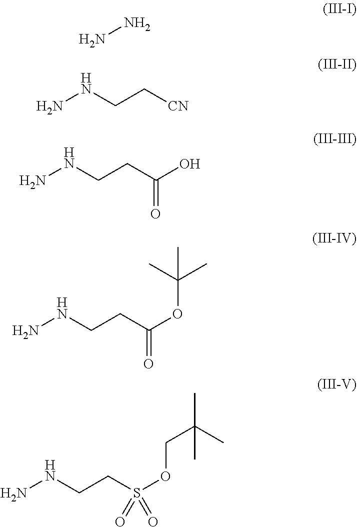

for preparing a compound of formula (I).

In another embodiment of the invention there is provided the use of a compound of formula (IV-b) (or a salt thereof) for preparing a compound of formula (I)

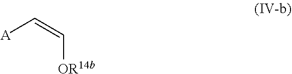

wherein A is as defined herein and R^{14b} is hydrogen or C₁-C₆alkyl.

Preferably, there is provided the use of a compound of formula (IV-b) (or a salt thereof) for preparing a compound of formula (I) wherein

A is selected from the group consisting of formula A-Ia to A-IIIa (preferably, A-Ia or A-IIIa) below,

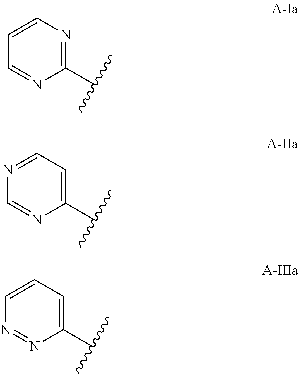 [SMILES: *c1ncccn1 ; *c1ccncn1 ; *c1cccnn1]

wherein the jagged line defines the point of attachment to the remaining part of a compound of formula (IV-b); and
R^{14b} is hydrogen.

More preferably, there is provided the use of a compound of formula (IV-Ib), (IV-IIb), (IV-IIIb), (IV-IVb), (IV-Vb), (IV-VIb), (IV-VIIb), (IV-VIIIb) or (IV-IXb) below

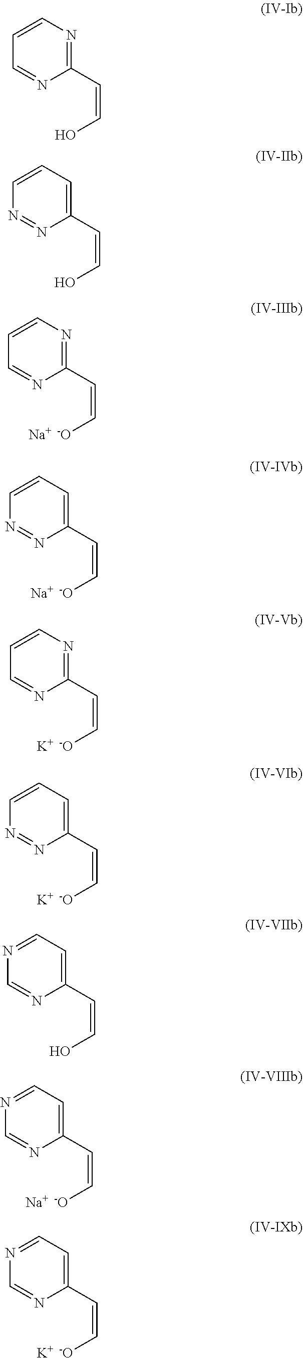

for preparing a compound of formula (I).

Even more preferably, there is provided the use of a compound of formula (IV-Ib), (IV-IIb), (IV-IIIb), (IV-IVb), (IV-Vb) or (IV-VIb) below

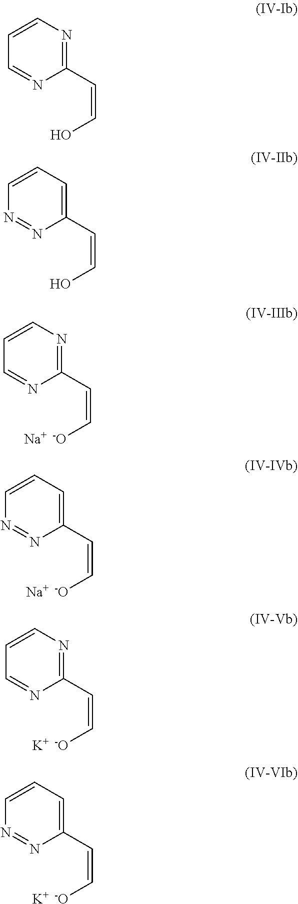

for preparing a compound of formula (I).

In another embodiment of the invention there is provided the use of a compound of formula (IV-c) for preparing a compound of formula (I)

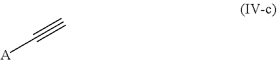

wherein A is as defined herein.

Preferably, there is provided the use of a compound of formula (IV-c) for preparing a compound of formula (I) wherein
A is selected from the group consisting of formula A-Ia to A-IIIa below,

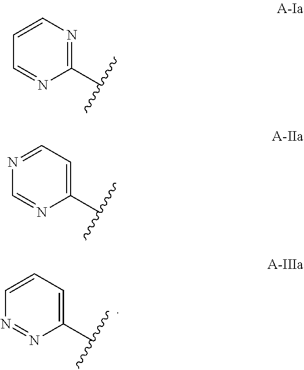 [SMILES: *c1ncccn1 ; *c1ccncn1 ; *c1cccnn1]

wherein the jagged line defines the point of attachment to the remaining part of a compound of formula (IV-c).

More preferably, there is provided the use of a compound of formula (IV-Ic) or (IV-IIc) below

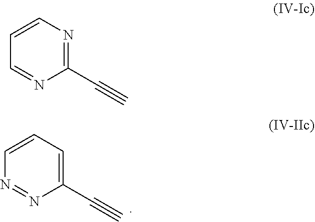

The present invention further provides a process as referred to above, wherein the compound of formula (II) is produced by:
reacting a compound of formula (IV)

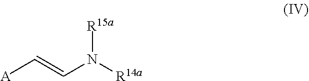

wherein A, R^{14a} and R^{15a} are as defined herein;
with a compound of formula (V)

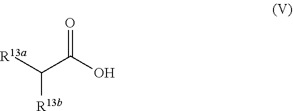

wherein each R^{13a} and R^{13b} are independently selected from the group consisting of halogen, —OR¹⁶ and —NR¹⁴R¹⁵ (preferably, preferably, each R^{13a} and R^{13b} are independently selected from the group consisting of —OH, morpholinyl, piperidinyl and pyrrolidinyl); or R^{13a} and R^{13b} together are ═O;
wherein R¹⁴, R¹⁵ and R¹⁶ are as defined herein, to produce a compound of formula (II)

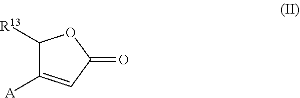

wherein A and R¹³ are as defined herein.

The present invention still further provides a process wherein the compound of formula (IV) is produced by:
reacting a compound of formula (VI)

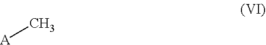

wherein A is as defined herein, with a compound of formula (VII)

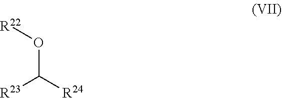

wherein R²² is C₁-C₆alkyl (preferably, methyl);
R²³ and R²⁴ are independently selected from the group consisting of C₁-C₆alkoxy and —NR²⁵R²⁶ (preferably, methoxy and N(Me)₂);
R²⁵ and R²⁶ are independently selected from C₁-C₆alkyl; or
R²⁵ and R²⁶ together with the nitrogen atom to which they are attached form a 4- to 6-membered heterocyclyl ring which optionally comprises one additional heteroatom individually selected from nitrogen, oxygen and sulfur;
and a compound of formula (VIII)

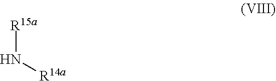

wherein R^{14a} and R^{15a} are as defined herein;
to produce a compound of formula (IV)

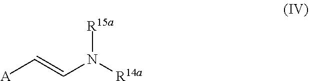

wherein A, R^{14a} and R^{15a} are as defined herein.

Scheme 1 below describes the reactions of the invention in more detail. The substituent definitions are as defined herein.

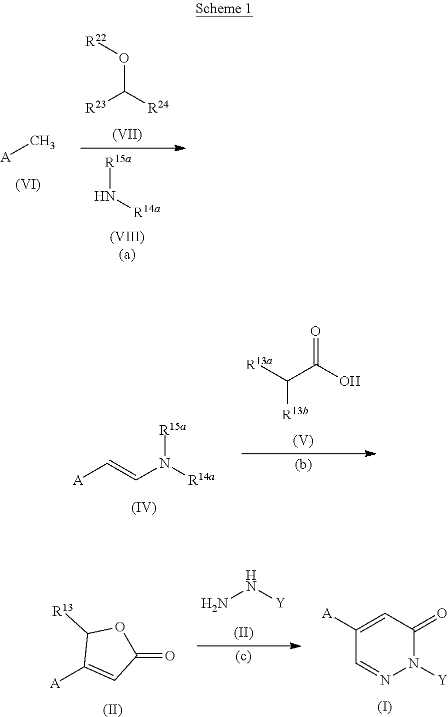

### Step (a) Formylation:

Compounds of formula (IV) can be prepared by reacting a compound of formula (VI)

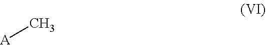

wherein A is as defined herein, with a compound of formula (VII)

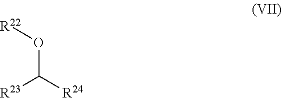

wherein R²², R²³ and R²⁴ are as defined herein;
and a compound of formula (VIII)

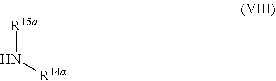

wherein R^{14a} and R^{15a} are as defined herein;
to produce a compound of formula (IV)

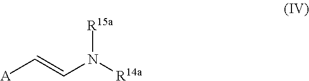

wherein A, R^{14a} and R^{15a} are as defined herein.

Typically the process described in step (a) is carried out in the presence of a catalytic amount of acid, or a catalytic mixture of acids, such as but not limited to, trifluoroacetic acid, acetic acid, benzoic acid, pivalic acid, propionic acid, butylated hydroxytoluene (BHT), 2,6-Di-tert-butylphenol, 2,4,6-Tri-tert-butylphenol, methanesulfonic acid, hydrochloric acid or sulfuric acid. Preferably, process step (a) is carried out in the presence of an acid with a non-alkylable anion, such as but not limited to butylated hydroxytoluene (BHT), 2,6-Di-tert-butylphenol or 2,4,6-Tri-tert-butylphenol.

The amount of acid is typically from 0.05 to 40 mol % (based on a compound of formula (VI)), preferably from 0.1 to 20 mol %.

The process described in step (a) may be carried out in the absence of a solvent, or in a solvent, or mixture of solvents, such as but not limited to, tetrahydrofuran, 2-methyltetrahydrofuran, diethylether, tert-butylmethylether, tert-amyl methyl ether, cyclopentyl methyl ether, dimethoxymethane, diethoxymethane, dipropoxy methane, 1,3-dioxolane, ethyl acetate, dimethyl carbonate, dichloromethane, dichloroethane, N,N-dimethylformamide, N,N-dimethylacetamide, N-methyl pyrrolidone (NMP), acetonitrile, propionitrile, butyronitrile, benzonitrile, toluene, 1,4-dioxane or sulfolane.

This step can be carried out at a temperature of from 0° C. to 230° C., preferably, from 150° C. to 230° C., more preferably from 180° C. to 220° C.

In another embodiment, this step can be carried out at a temperature of from 50° C. to 110° C.

The skilled person would appreciate that unreacted starting material, a compound of formula (VI), (VII) or (VIII) can be recovered and reused.

Preferably, this step is carried out in a closed vessel (for example but not limited to an autoclave).

Preferably, this step is carried out with the continuous removal (for example, but not limited, by fractional distillation under pressure) of by-products (for example methanol and/or ethanol). More preferably, wherein a compound of formula (VII) is trimethyl orthoformate or triethyl orthoformate the reaction is carried out with the continuous removal of methanol (when trimethyl orthoformate is used) or ethanol (when triethyl orthoformate is used).

### Step (b) Furanone Formation:

Compounds of formula (II) can be produced by reacting a compound of formula (IV)

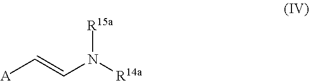

wherein A, R^{14a} and R^{15a} are as defined herein, with a compound of formula (V)

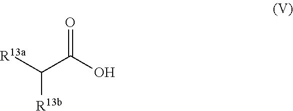

wherein each R^{13a} and R^{13b} are as defined herein, to produce a compound of formula (II)

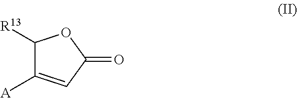

wherein A and R¹³ are as defined herein.

Typically, the process described in step (b) is carried out in the presence of an acid, or mixture of acids, such as hydrochloric acid, sulfuric acid, chloroacetic acid, trichloroacetic acid, propionic acid, acetic acid, acetic anhydride, formic acid, n-butanoic acid, n-pentanoic acid, n-hexanoic acid and propionic anhydride. More preferably, process step (b) is carried out in the presence of acetic acid and/or formic acid.

Typically the process described in step (b) is carried out in a solvent, or mixture of solvents, such as but not limited to, water, acetonitrile, propionitrile, methanol, iso-Amyl alcohol, isopropanol, t-Butanol t-amyl alcohol, N,N-dimethylformamide, N,N-dimethylacetamide, N-methyl pyrrolidone (NMP), acetic acid and propionic acid.

This step of the reaction can be carried out at a temperature of from −78° C. to 120° C., preferably, from −20° C. to 60° C. More preferably, from −10° C. to 30° C.

### Step (c) Ring Expansion:

The compound of formula (I) can be prepared by reacting a compound of formula (II):

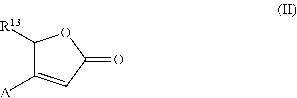

wherein A and R¹³ are as defined herein, with a compound of formula (III):

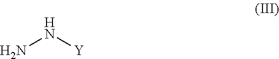

wherein Y is as defined herein, to give a compound of formula (I);

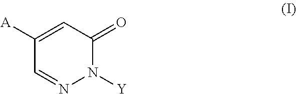

wherein A and Y are as defined herein.

Typically in this process step (c) is carried out in the presence of an acid, or mixture of acids, such as hydrochloric acid, sulfuric acid, chloroacetic acid, trichloroacetic acid, propionic acid, acetic acid, acetic anhydride, formic acid, n-butanoic acid, n-pentanoic acid, n-hexanoic acid and propionic anhydride. More preferably, process step (c) is carried out in the presence of acetic acid and/or trifluoroacetic acid.

Typically the process described in step (c) is carried out in a solvent, or mixture of solvents, such as but not limited to, alcohols (such as MeOH, iPrOH, EtOH, BuOH, tBuOH, tert amyl alcohol), tetrahydrofuran, 2-methyltetrahydrofuran, diethylether, tert-butylmethylether, tert-amyl methyl ether, cyclopentyl methyl ether, dimethoxymethane, diethoxymethane, dipropoxy methane, 1,3-dioxolane, ethyl acetate, dimethyl carbonate, dichloromethane, dichloroethane, N,N-dimethylformamide, N,N-dimethylacetamide, N-methyl pyrrolidone (NMP), acetonitrile, propionitrile, butyronitrile, benzonitrile, 1,4-dioxane, sulfolane, acetic acid and propionic acid. Preferably, the process described in step (c) is carried out in a solvent, or mixture of solvents selected from the group consisting of MeOH, iPrOH, EtOH, BuOH, tBuOH and tert amyl alcohol.

This step of the reaction can be carried out at a temperature of from −78° C. to 120° C., preferably, from −20° C. to 80° C. More preferably, from −10° C. to 60° C.

The skilled person would appreciate that the temperature of the process according to the invention can vary in each of steps (a), (b) and (c). Furthermore, this variability in temperature may also reflect the choice of solvent used.

Preferably, the process of the present invention is carried out under an inert atmosphere, such as nitrogen or argon.

Scheme 2 below shows an additional alkylation step (d) which may be carried out when in a compound of formula (I), Y is hydrogen.

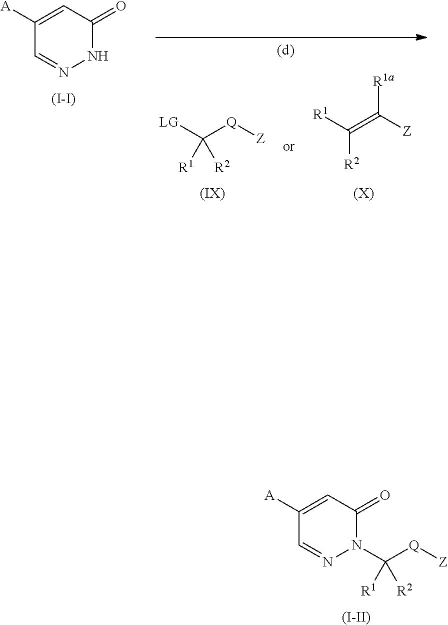

### Step (d) Alkylation:

Compounds of formula (I-II) can be prepared by reacting a compound of formula (I-I)

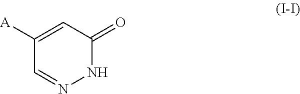

wherein A is as defined herein for the compound of formula (I) with a suitable alkylating agent to give a compound of formula (I-II)

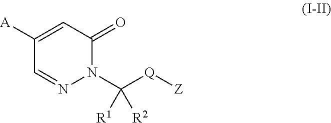

wherein A, R¹, R², Q and Z are as defined herein for compounds of formula (I).

Typically in this process of the invention such suitable alkylating agents may comprise a suitable leaving group (compounds of formula (IX)), for example these may include but are not limited to bromoacetic acid, methyl bromoacetate, 3-bromopropionoic acid, methyl 3-bromopropionate, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide and 3-chloro-2,2-dimethyl-propanoic acid. Alternatively the alkylating agent used in a process of the invention may be a suitably activated electrohphilic alkene (compounds of formula (X), for example these may include but are not limited to acrylic acid, methacrylic acid, acrylonitrile, crotonic acid, 3,3-dimethylacrylic acid, methyl acrylate, ethyl acrylate, tert-butyl acrylate, ethene sulfonic acid, isopropyl ethylenesulfonate and 2,2-dimethylpropyl ethenesulfonate.

Preferably, the suitable alkylating agent is either a compound of formula (IX) or formula (X)

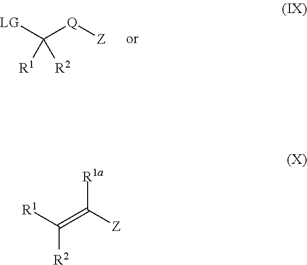

wherein R¹, R², R^{1a}, Q and Z are as defined herein for compounds of formula (I) and LG is a suitable leaving group (preferably, chloro, bromo or trifluoromethanesulfonate).

More preferably, the suitable alkylating agent is a compound of formula (X)

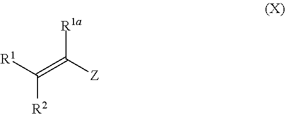

wherein, R¹, R², R^{1a} and Z are as defined above for compounds of formula (I).

Even more preferably, the suitable alkylating agent is selected from the group consisting of acrylonitrile, ethyl acrylate and tert-butyl acrylate.

Typically this process step (d) is carried out in the presence of a base, or mixture of bases, such as potassium carbonate, sodium carbonate, potassium hydroxide, sodium hydroxide, triethylamine, tripropylamine, tributylamine, pyridine. More preferably, process step (d) is carried out in the presence of potassium carbonate.

Typically this process step (d) is carried out in the presence of a phase transfer catalyst such as Tricaprylmethylammonium chloride, Benzyl Tributyl Ammonium Bromide, Benzyl Tributyl Ammonium Chloride, Benzyl Triethyl Ammonium Bromide, Benzyl Triethyl Ammonium Chloride, Benzyl Trimethyl Ammonium Chloride, Cetyl Pyridinium Bromide, Cetyl Pyridinium Chloride, Cetyl Trimethyl Ammonium Bromide, Didecyl Dimethyl Ammonium Chloride, Dimethyl Distearyl Ammonium Chloride, Dodecyl Trimethyl Ammonium Bromide, Dodecyl Trimethyl Ammonium Chloride, Hexadecyl Trimethyl Ammonium Chloride, Methyl Tributyl Ammonium Chloride, Methyl Tricaprylyl Ammonium Chloride, Methyl Trioctyl Ammonium Chloride, Phenyl Trimethyl Ammonium Chloride, Tetrabutyl Ammonium Bromide, Tetrabutyl Ammonium Chloride, Tetrabutyl Ammonium Fluoride, Tetrabutyl Ammonium Hydrogen Sulfate, Tetrabutyl Ammonium Hydroxide, Tetrabutyl Ammonium Iodide, Tetraethyl Ammonium Bromide, Tetraethyl Ammonium Chloride, Tetraethyl Ammonium Hydroxide, Tetramethyl Ammonium Bromide, Tetramethyl Ammonium Chloride, Tetraoctyl Ammonium Bromide, Tetrapropyl Ammonium Bromide, Tetrapropyl Ammonium Hydroxide, Triethyl Benzyl Ammonium Chloride. Preferably, process step (d) is carried out in the presence of Triethyl Benzyl Ammonium Chloride, Benzyl Triethyl Ammonium Bromide or Tetrabutyl Ammonium Bromide.

Typically the process described in step (d) is carried out by stirring a compound of formula (I-I) with an alkylating agent of formula (IX) or (X) in a solvent, or mixture of solvents, such as acetone, dichloromethane, dichloroethane, N,N-dimethylformamide, acetonitrile, tetrahydrofuran, 2-methyltetrahydrofuran, 1,4-dioxane, water, acetic acid or trifluroacetic acid.

The reaction can be carried out at a temperature of from −78° C. to 150° C., preferably, from 20° C. to 100° C.

In a preferred embodiment of the invention the compound of formula (I) (which can be depicted as a compound of formula (I-I) or (I-II)) is further converted (for example via a sulfurization, desulfurization, hydrolysis, and/or a salt exchange as shown in scheme 3 below) to give an agronomically acceptable salt of formula (Ia) or a zwitterion of formula (Ib),

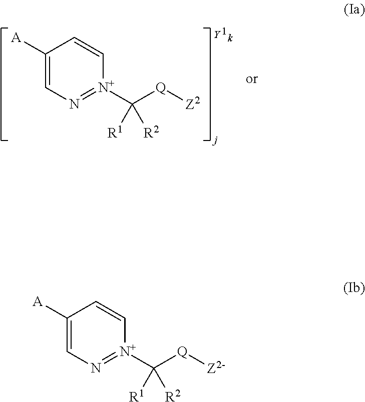

wherein Y¹ represents an agronomically acceptable anion and j and k represent integers that may be selected from 1, 2 or 3 (preferably, Y¹ is Cl⁻ and j and k are 1), and A, R¹, R² and Q are as defined herein and Z² is —C(O)OH or —S(O)₂OH (the skilled person would appreciate that Z²⁻ represents —C(O)O⁻ or —S(O)₂O⁻).

More preferably, the compound of formula (I) is further converted to give a compound of formula (Ia),

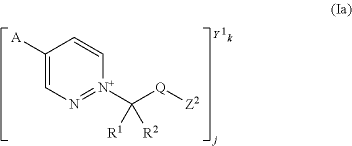

wherein Y¹ represents an agronomically acceptable anion and j and k represent integers that may be selected from 1, 2 or 3 (preferably, Y¹ is Cl⁻ and j and k are 1), and A, R¹, R² and Q are as defined herein and Z² is —C(O)OH.

Preferably, in a compound of formula (Ia) Y¹ is chloride, bromide, iodide, hydroxide, bicarbonate, acetate, trifluoroacetate, methylsulfate, tosylate, benzoate and nitrate, wherein j and k are 1. More preferably, in a compound of formula (Ia) Y¹ is Cl⁻ and j and k are 1.

Scheme 3 below shows how the compound of formula (I) is further converted to a compound of formula (Ia) or (Ib).

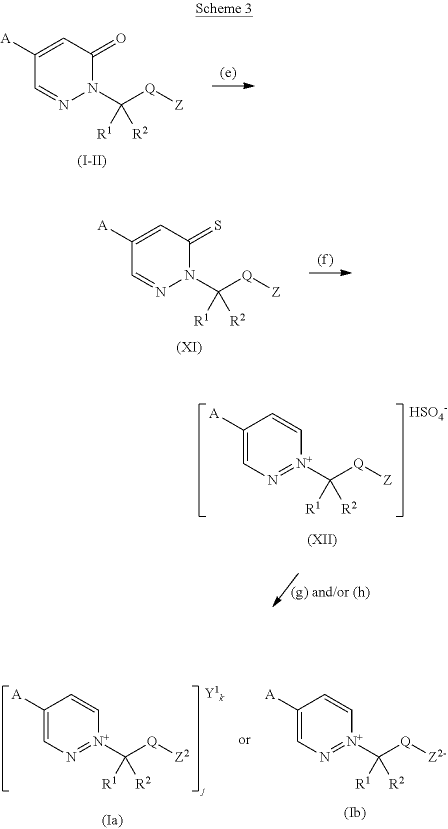

### Step (e) Sulfurization:

The compound of formula (XI) is can be prepared by reacting a compound of formula (I-II):

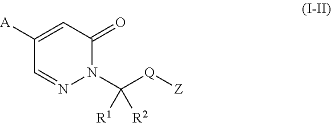

wherein A, R¹, R², Q and Z are as defined herein, with a sulfurizing agent to give a compound of formula (XI)

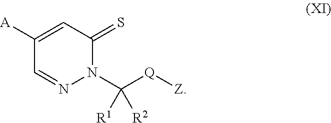

Typically in this process step (e) examples of such sulfurizing agents include but are not limited to, phosphorous pentasulfide (P₂S₅) and lawesson's reagent (2,4-Bis(4-methoxyphenyl)-2,4-dithioxo-1,3,2,4-dithiadiphosphetane). Preferably, the sulfurizing agent is phosphorous pentasulfide.

Typically the process described in step (e) is carried out by stirring a compound of formula (I-II) with a sulfurizing agent in a solvent, or mixture of solvents, such as chlorobenzene or pyridine.

The reaction can be carried out at a temperature of from 20° C. to 150° C., preferably from 60° C. to 120° C.

Preferably process step (c) of the present invention is carried out under an inert atmosphere, such as nitrogen or argon.

### Step (f) Desulfurization:

Compounds of formula (XII)

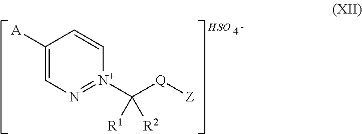

wherein A, R¹, R², Q and Z are as defined above, can be prepared by, reacting a compound of formula (XI):

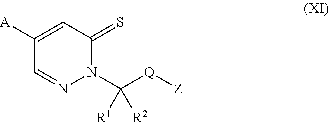

wherein A, R¹, R², Q and Z are as defined above for compound of formula (I), in a suitable reaction medium comprising a desulfurization agent, to give a compound of formula (XII).

The process step (f is typically carried out in a suitable reaction medium, which can be a solvent which is in principle any solvent or mixture of solvents that are inert under the reaction conditions.

The process step (f is typically carried out in solvent or mixture of solvents such as but not limited to, for example, water, acetonitrile, propanenitrile, formamide, dimethyl formamide, N-methylformamide, dimethyl sulphoxide, N-methyl pyrrolidone (NMP), dimethyl acetamide, 1,3-Dimethyl-2-imidazolidinone, sulfolane, N-butylpyrrolidone (NBP), N-octylpyrrolidone, cyclohexane, pentane, 2-methylpentane, n-hexane, isooctane, methyl cyclohexane, heptane, methylcyclopentane, petroleum spirit, cis-decalin, n-octane, nonane, decane, limonene, trifluorotoluene, chlorobenzene, 1,2-dichlorobenzene, 1,2,4-trichlorobenzene, 1,1-dichloroethane, 1,1,1-trichloroethane, trichloroethylene, bromobenzene, 1-chlorobutane, perfluoromethylcyclohexane, iodobenzene, dichloromethane, chloroform, perfluorohexane, 1,2-dichloroethane, perfluorotoluene, perfluorocyclohexane, chloroacetic acid, trichloroacetic acid, propionic acid, acetic acid, acetic anhydride, formic acid, n-butanoic acid, n-pentanoic acid, n-hexanoic acid, propionic anhydride, methyl acetate, dimethyl carbonate, ethyl acetate, ethyl formate, isopropyl acetate, propyl acetate, methyl lactate, ethyl propionate, t-butyl acetate, ethylene carbonate, propylene carbonate, butyl acetate, ethyl lactate, n-octyl acetate, diethyl carbonate, iso-butylacetate, formic acid methyl ester, butyrolactone, methyl benzoate, dimethyl phthalate, ethyl benzoate, i-pentyl acetate, methyl propionate, butyronitrile, N,N-diethylacetamide, tetraethylurea, N,N-diethylpropionamide, valeronitrile, malononitrile, tetramethylurea, N,N-dimethyltrifluoroacetamide, N,N-dimethylchloroacetamide, di-n-butyl sulfoxide, N,N-diethylbenzamide, toluene, xylene iso-mix, cumene, isopropylbenzene, p-xylene, mesitylene, benzonitrile, nitrobenzene, o-xylene, m-xylene, ethylbenzene, tetralin, methanol, iso-Amyl alcohol, isopropanol, t-Butanol and t-amyl alcohol.

Typically process step (f) is carried out in the presence of an acid. Preferably the acid is selected from the group consisting of chloroacetic acid, trichloroacetic acid, propionic acid, acetic acid, acetic anhydride, formic acid, n-butanoic acid, n-pentanoic acid, n-hexanoic acid and propionic anhydride. More preferably, the acid is acetic acid or formic acid.

Preferably, the desulfurization agent is an oxidant. In principle any oxidation reagent known to a person skilled in the art for oxidation of an organic sulfide group could be employed.

Suitable oxidizing agents include, but are not limited to, hydrogen peroxide, hydrogen peroxide and a suitable catalyst (for example, but are not limited to: TiCl₃, Mn(OAc)₃.2H₂O and a bipyridine ligand, VO(acac)₂ and a bidentate ligand, Ti(OiPr₄) and a bidentate ligand, Polyoxymetalates, Na₂WO₄ together with additives such as PhPO₃H₂ and CH₃(n-C₈H₁₇)₃NHSO₄, lanthanide catalysts such as Sc(OTf)₃, organic molecules can also act as catalysts, for example flavins), chlorine, with or without a suitable catalyst (as listed above), bromine with or without a suitable catalyst (as listed above), organic hydroperoxides (for example peracetic acid, performic acid, t-Butylhydroperoxide, cumylhydroperoxide, MCPBA), an organic hydroperoxide prepared in situ (for example from the reaction of H₂O₂ and a carboxylic acid+a suitable catalyst), organic peroxides (for example benzoyl peroxide, or di-terbutylperoxide), amine N-oxides (for example N-Methylmorpholine Oxide, pyridine N-oxide or triethylamine N-oxide peroxide derivative), inorganic oxidants (NaIO₄, KMnO₄, MnO₂, CrO₃), inorganic oxidants prepared in situ (for example, a Ru catalyst+an oxidant forms in situ RuO4 which maybe a capable oxidant), inorganic hydroperoxides, inorganic peroxides, dioxiranes (for example DMDO), oxone, oxygen (oxygen+a suitable catalyst such as NO₂, or Cerric ammonium nitrate), air+a suitable catalyst (such systems can lead to the in-situ formation of peroxides and suitable catalysts can be for example, but not limited to, Fe(NO₃)₃—FeBr₃), NaOCl (which may be used in conjunction with catalytic amounts of a stable radical such as (2,2,6,6-tetramethylpiperidin-1-yl)oxyl (TEMPO), 4-hydroxy-TEMPO or 4-acetylamino-TEMPO, optionally catalytic amounts of sodium bromide may also be added), NaOBr, HNO₃, biocatalysts such as peroxidases and monooxygenases and nitrosyl chloride (prepared in situ).

Preferably, the desulfurization agent is a peroxide or derivative thereof (for example peracetic acid, performic acid, t-Butylhydroperoxide, cumylhydroperoxide, MCPBA). Most preferably, the desulfurization agent is hydrogen peroxide.

The skilled person would appreciate that the temperature of the process according to the invention can vary depending on the choice of solvent used. Typically, the process according to the invention is carried out at a temperature from 40° C. to 120° C., preferably from 80° C. to 110° C.

### Step (q) Hydrolysis:

The hydrolysis can be performed using methods known to a person skilled in the art. The hydrolysis is typically performed using a suitable reagent, including, but not limited to aqueous sulfuric acid, concentrated hydrochloric acid or an acidic ion exchange resin.

Typically, the hydrolysis is carried out using aqueous hydrochroric acid, optionally in the presence of an additional suitable solvent, at a suitable temperature from 0° C. to 120° C. (preferably, from 20° C. to 100° C.).

### Step (h) Salt Exchange:

The salt exchange of a compound of formula (XII) to a compound of formula (Ia) can be performed using methods known to a person skilled in the art and refers to the process of converting one salt form of a compound into another (anion exchange), for example coverting a hydrogen sulfate (HSO₄⁻) salt to a chloride (Cl⁻) salt. The salt exchange is typically performed using an ion exchange resin or by salt metathesis. Salt metathesis reactions are dependent on the ions involved, for example a compound of formula (XII) wherein the agronomically acceptable salt is a hydrogen sulfate anion (HSO₄⁻) may be switched to a compound of formula (Ia) wherein Y¹ is a chloride anion (Cl⁻) by treatment with an aqueous solution of barium chloride (BaCl₂) or calcium chloride (CaCl₂). Preferably, the salt exchange of a compound of formula (XII) to a compound of formula (Ia) is performed with barium chloride.

In a preferred embodiment of the invention there is provided a process for the preparation of a compound of formula (I):

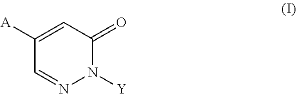

wherein
A is a 6-membered heteroaryl selected from the group consisting of formula A-Ia to A-IIIa (preferably A-Ia or A-IIIa) below

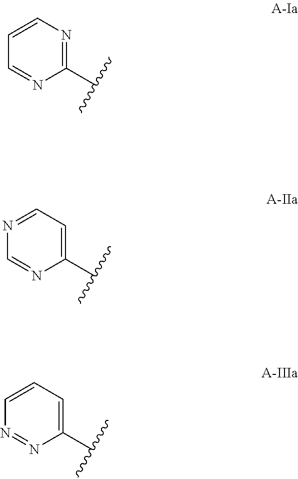 [SMILES: *c1ncccn1 ; *c1ccncn1 ; *c1cccnn1]

wherein the jagged line defines the point of attachment to the remaining part of a compound of formula (I); and
Y is hydrogen or the group Y-I below

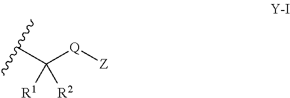

wherein the jagged line defines the point of attachment to the remaining part of a compound of formula (I); and
R¹ is hydrogen;
R² is hydrogen;
Q is (CR^{1a}R^{2b})ₘ;
m is 1;
each R^{1a} and R^{2b} are hydrogen;
Z is selected from the group consisting of —CN, —CH₂OH, —C(O)OR¹⁰, and —S(O)₂OR¹⁰ (preferably —CN, —C(O)OR¹⁰, and —S(O)₂OR¹⁰); and
R¹⁰ is selected from the group consisting of hydrogen and C₁-C₆alkyl (preferably, hydrogen, methyl, ethyl, iso-propyl, 2,2-dimethylpropyl and tert-butyl);
said process comprising:
reacting a compound of formula (II):

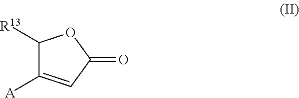

wherein A is as defined above;
R¹³ is selected from the group consisting of R¹³ is selected from the group consisting of chloro, —OH, —OMe, —OEt, —N(Me)₂, morpholinyl, piperidinyl and pyrrolidinyl (preferably, —OH, —N(Me)₂, morpholinyl, piperidinyl and pyrrolidinyl);
with a compound of formula (III):

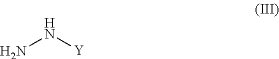

wherein Y is as defined above, to produce a compound of formula (I);

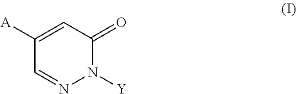

wherein A and Y are as defined above.

### EXAMPLES

The following examples further illustrate, but do not limit, the invention. Those skilled in the art will promptly recognise appropriate variations from the procedures both as to reactants and as to reaction conditions and techniques.

The following abbreviations are used: s=singlet; br s=broad singlet; d=doublet; dd=double doublet; dt=double triplet; t=triplet, tt=triple triplet, q=quartet, quin=quintuplet, sept=septet; m=multiplet; GC=gas chromatography, RT=retention time, Ti=internal temperature, MH*=molecular mass of the molecular cation, M=molar, Q¹HNMR=quantitative ¹HNMR, RT=room temperature, UFLC=Ultra-fast liquid chromatography.

¹H NMR spectra are recorded at 400 MHz unless indicated otherwise and chemical shifts are recorded in ppm.

### LCMS Methods:

### Standard:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85

### Standard Long:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85

### Example 1: Preparation of 2-[(2-Pyrrolidin-1-ylvinyl]pyrimidine

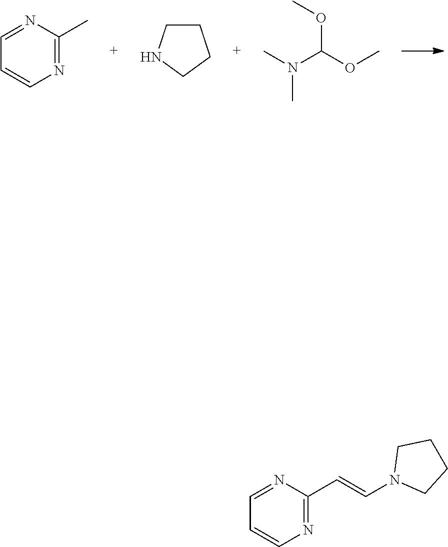

A mixture of 2-methyl-pyrimidine (10 g, 0.1063 mol), pyrrolidine (15.2 g, 0.2125 mol) and N,N-dimethylformamide dimethyl acetal (26.1 g, 0.2125 mol) was heated at 87° C. (internal temperature) for 15 h. After cooling down to room temperature, the mixture was concentrated under vacuum to give a yellowish solid. 300 ml of tButyl-methyl-ether were added to this solid, and it was dissolved at reflux. The solution was then cooled down to 0° C., stirred for 20 minutes, the solid was filtered, washed once with cold tButyl-methyl-ether, collected and dried under high vacuum. 12.3 g of 2-[(E)-2-pyrrolidin-1-ylvinyl] pyrimidine, a white solid, pure at 97% w/w as measured by Quantitative NMR was obtained. The filtrate was concentrated under vacuum and 200 ml of tButyl-methyl-ether was added. After full dissolution was achieved at reflux, the solution was then cooled down to 0° C., stirred for 20 minutes, the solid was filtered, washed once with cold tButyl-methyl-ether, collected and dried under high vacuum. 4.7 g of 2-[2-pyrrolidin-1-ylvinyl]pyrimidine, a white solid, pure at 94% w/w as measured by Quantitative NMR was obtained. The two batches were combined to deliver 17 g of the title compound, pure at 96% w/w (84.1% yield).

1H NMR (400 MHz, CDCl3) δ ppm 1.85-2.05 (m, 4H) 3.28-3.44 (m, 4H) 5.25 (d, 1H) 6.67 (t, 1H) 7.99 (d, 1H) 8.38 (d, 2H).

### Example 2: Preparation of 4-[2-pyrimidin-2-ylvinyl]morpholine

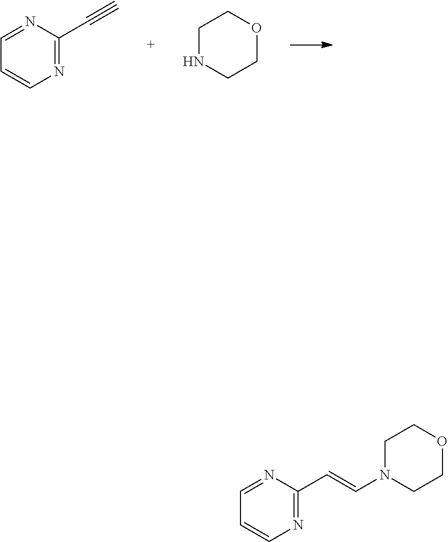

A mixture of 2-ethynylpyrimidine (0.25 g, 2.33 mmol) and morpholine (0.43 g, 4.89 mmol) was heated at 100° C. for 20 minutes. The mixture was then cooled down to room temperature and concentrated under vacuum. The crude title compound was obtained as an orange oil which solidified on standing (0.553 g) with a purity of 75% w/w as measured by Quantitative NMR. Most of the contaminant was residual morpholine.

1H NMR (400 MHz, CDCl3) δ ppm 3.23-3.33 (m, 4H) 3.74-3.79 (m, 4H) 5.49 (d, J=13.57 Hz, 1H) 6.78 (t, J=4.95 Hz, 1H) 7.66 (d, J=13.20 Hz, 1H) 8.44 (d, J=4.77 Hz, 2H)

### Example 3: Preparation of 2-[2-(1-piperidyl)vinyl]pyrimidine

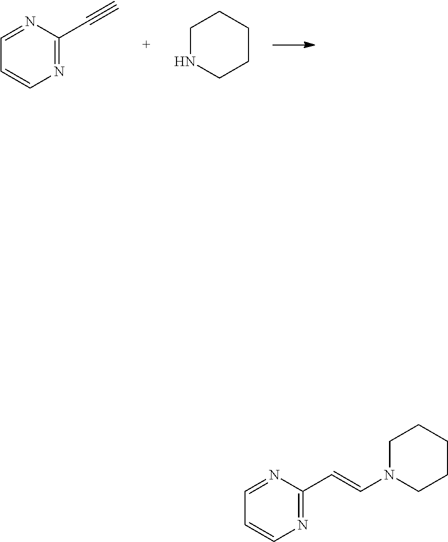

A mixture of 2-ethynylpyrimidine (0.25 g, 2.33 mmol) and piperidine (4.89 mmol) was heated at 100° C. for 20 minutes. The mixture was then cooled down to room temperature and concentrated under vacuum. The crude title compound was obtained.

1H-NMR (400 MHz, THF-d8) δ ppm 8.37 (d, J=4.77 Hz, 2H), 7.76 (d, J=13.57 Hz, 1H), 6.70 (t, J=4.77 Hz, 1H), 5.43 (d, J=13.20 Hz, 1H), 3.19-3.30 (m, 4H), 1.56-1.67 (m, 6H)

### Example 4: Preparation of 2-morpholino-3-pyrimidin-2-yl-2H-furan-5-one from 2-[(2-pyrrolidin-1-ylvinyl]pyrimidine

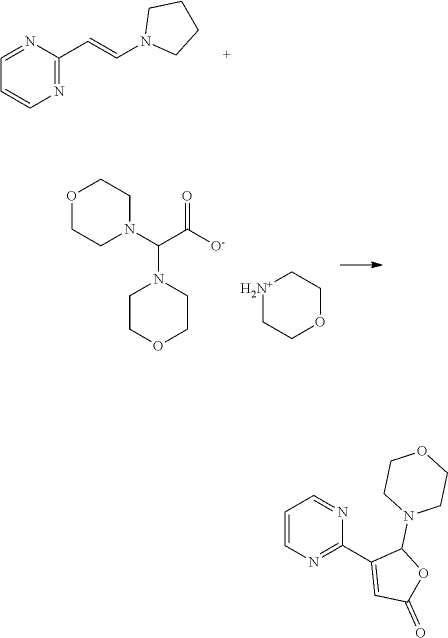 [SMILES: C(=C\N1CCCC1)/c1ncccn1 ; O=C([O-])C(N1CCOCC1)N1CCOCC1 ; C1COCC[NH2+]1 ; O=C1C=C(c2ncccn2)C(N2CCOCC2)O1]

To a cold (1° C.) solution of 2-[(E)-2-pyrrolidin-1-ylvinyl]pyrimidine (pure at 96% w/w) (5.04 g, 27.6 mmol) and morpholin-4-ium 2,2-dimorpholinoacetate (1.2 eq, 33.1 mmol) was added acetic acid (5 eq, 138 mmol) dropwise over a period of 25 minutes. The cold temperature (1° C.) was maintained for another 15 minutes before the solution allowed to warm at room temperature and stirred for 2 h30. The solution was then cooled down to 2° C. and stirred for another 30 minutes. The resulting suspension was then filtered, the solid was washed with cold (5° C.) methanol (12.5 ml) twice, collected and dried under reduced pressure until constant weight (5.76 g). The title compound was thus obtained as a white solid with a purity of 98% w/w as measured by Quantitative NMR (82.5% yield).

1H NMR (400 MHz, CDCl3) δ ppm 2.75-2.88 (m, 4H) 3.53-3.69 (m, 4H) 6.27 (d, J=1.4 Hz, 1H) 6.94 (d, J=1.4 Hz, 1H) 7.34 (t, J=4.7 Hz, 1H) 8.85 (d, J=4.7 Hz, 2H)

### Example 5: Preparation of 3-pyrimidin-2-yl-2-pyrrolidin-1-yl-2H-furan-5-one and 2-hydroxy-3-pyrimidin-2-yl-2H-furan-5-one from 2-[(2-pyrrolidin-1-ylvinyl]pyrimidine

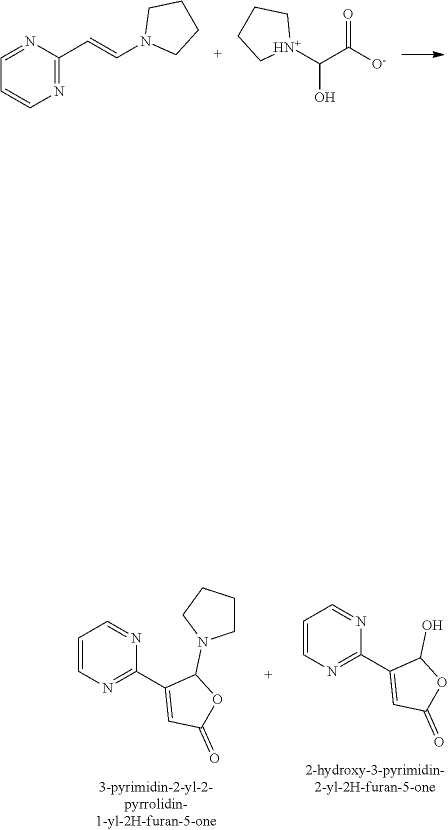

### Preparation of 2-hydroxy-2-pyrrolidin-1-ium-1-yl-acetate

To a solution of glyoxylic acid monohydrate (4 g, 42.1 mmol, 1.0 eq.) in ethanol (8.6 mL). The resulting solution was cooled to 0° C. A solution of a solution of Pyrrolidine (1.05 equiv., 44.2581 mmol, 99.5 mass %) in ethanol (1.7 mL) was added dropwise at 0° C. After addition reaction was stirred at 0° C. for 2 h. The resulting beige solid which had formed was filtered, washed with Et2O (3×) and dried to give white crystals (3.3 g) of 2-hydroxy-2-pyrrolidin-1-yl-acetic acid which was used as such.

A vial was charged at room temperature with 2-[2-pyrrolidin-1-ylvinyl]pyrimidine (0.3 g, 1.71 mmol, 1 eq.) and 2-hydroxy-2-pyrrolidin-1-ium-1-yl-acetate (0.271 g, 1.89 mmol, 1.1 eq.) and then dissolved in methanol (2.57 mL, 1.5 mL/mmol). To this solution was added acetic acid (0.49 mL, 5 eq) dropwise over a period of 10 min then the reaction mixture was stirred for 2.5 h at rt. The solvent was carefully removed by using a pipette after settling of the reaction mixture. The resulting solid was washed with Et2O (3×5 mL). Et2O removed with pipette. The remaining solid was then dried under reduced pressure to yielding the hydrolyzed product (2-hydroxy-3-pyrimidin-2-yl-2H-furan-5-one) as a yellow solid (49.4 mg).

2-hydroxy-3-pyrimidin-2-yl-2H-furan-5-one: 1H NMR (400 MHz, D6-DMSO) δ ppm 6.66 (dd, 1H) 6.94 (d, 1H) 7.60 (t, 1H) 8.06 (d, 1H) 8.99 (d, J=5.14 Hz, 2H)

Filtrate was concentrated then submitted to a separation on Normal Phase chromatography (Isco Combiflash) to give the title compound as a white solid (43.5 mg). Chromatography as purification technique does lead to partial formation of 2-hydroxy-3-pyrimidin-2-yl-2H-furan-5-one as well.

3-pyrimidin-2-yl-2-pyrrolidin-1-yl-2H-furan-5-one: 1H NMR (400 MHz, d6-DMSO) δ ppm 1.65 (br t, 4H) 2.60-2.78 (m, 4H) 6.62 (d, 1H) 6.90 (d, 1H) 7.58 (t, 1H) 8.97 (d, 2H)

### Example 6: Preparation of 2-(1-piperidyl)-3-pyrimidin-2-yl-2H-furan-5-one from 2-[2-(1-piperidyl)vinyl]pyrimidine

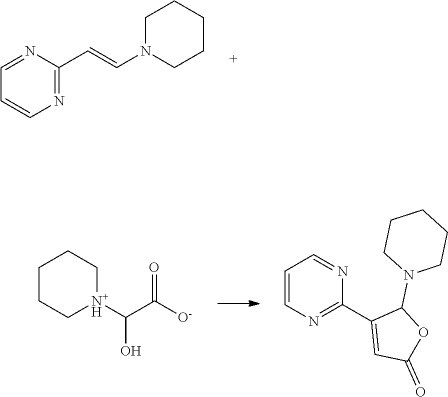

### Preparation of 2-hydroxy-2-piperidin-1-ium-1-yl-acetate

To a solution of glyoxylic acid monohydrate (5.00 g, 53.23 mmol, 1 eq.) in Toluene (40 mL).

Piperidine (4.65 g, 54.11 mmol, 1.02 eq.) was added drop-wise with vigorous stirring. The reaction vessel was stoppered & placed in freezer overnight. The crystals were collected by vacuum filtration and the cake was filtered. The cake was washed with Et2O (2×25 mL) to give 2-hydroxy-2-piperidin-1-ium-1-yl-acetate (8.1814 g, 50.7 mmol, 95.3% yield) as fine white powder which was used as such.

A 100 ml 3 necked RBF was charged with 2-(1-piperidyl)-3-pyrimidin-2-yl-2H-furan-5-one (0.20 g, 1.05 mmol, 1 eq.) and 2-hydroxy-2-piperidin-1-ium-1-yl-acetate (0.184 g, 1.16 mmol, 1.1 eq) and dissolved in methanol (1.5 mL/mmol) then acetic acid (0.32 g, 5.3 mmol, 5 eq) was added dropwise and then the reaction was stirred for 3 h at rt. The solvent was carefully removed by using a pipette after settling of the reaction mixture. The resulting solid was washed with Et2O (3×5 mL). Et2O removed with pipette. The remaining solid was then dried under reduced pressure to yielding the desired product and traces of hydrolyzed product as a yellow solid (46.1 mg). Filtrate was concentrated then submitted to a separation on Normal Phase chromatography (Isco Combiflash) to give the title compound as a white solid (43.5 mg).

2-(1-piperidyl)-3-pyrimidin-2-yl-2H-furan-5-one: 1H NMR (400 MHz, D6-DMSO) δ ppm 1.19-1.47 (m, 6H) 2.55-2.74 (m, 4H) 6.41 (d, 1H) 6.92 (d, 1H) 7.57-7.62 (m, 1H) 8.95-9.01 (m, 2H)

### Example 7: Preparation of 2-hydroxy-3-pyrimidin-2-yl-2H-furan-5-one from 2-methylpyrimidine

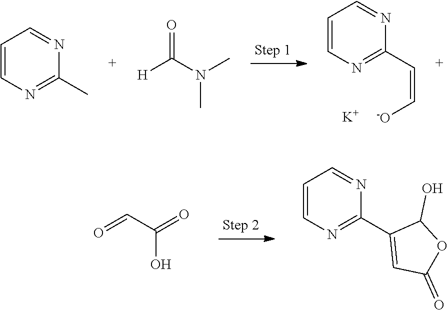

### Step 1:

Potassium tert-butoxide (2.50 eq, 1.45 g, 12.5 mmol, 2.5 eq.) was dissolved in N,N-dimethylformamide (7.50 mL) then 2-methylpyrimidine (0.48 g, 5.00 mmol, 1 eq.) was added and then the resulting mass was stirred at room temperature for 12 h. Potassium 2-pyrimidin-2-ylethenolate was used without any work up or determination of yield for step 2.

### Step 2:

A 50 ml three neck round bottom flask equipped with a thermometer, a gas inlet, a bubbler and a septum, was charged with glyoxylic acid monohydrate (0.61 g, 6.50 mmol, 1.30 eq) and dissolved with methanol (10.0 mL). The resulting reaction mixture was cooled down to −5° C. and acetic acid (2.87 mL, 50.0 mmol, 10 eq) was added then a solution of Potassium 2-pyrimidin-2-ylethenolate (from step 1) in Methanol (2 ml) was added dropwise at −5° C. Reaction temperature was maintained under 0° C. during the addition. Reaction mixture was allowed to warm to room temperature and stirred for 2 h. The reaction mixture was evaporated to dryness to give 7.2 g of the title compound as a black liquid (contained unquantified amounts of acetic acid and DMF). NMR and LC-MS consistent with the structure of desired product (7.2 g, 9% strength (determined by quant 1H NMR), 73% yield).

1H NMR (400 MHz, D6-DMSO) δ ppm 6.66 (dd, J=8.25, 0.92 Hz, 1H) 6.94 (d, J=0.73 Hz, 1H) 7.60 (t, J=4.95 Hz, 1H) 8.06 (d, J=8.44 Hz, 1H) 8.99 (d, J=5.14 Hz, 2H)

### Example 8: Preparation of 2-methoxy-3-pyrimidin-2-yl-2H-furan-5-one from 2-hydroxy-3-pyrimidin-2-yl-2H-furan-5-one

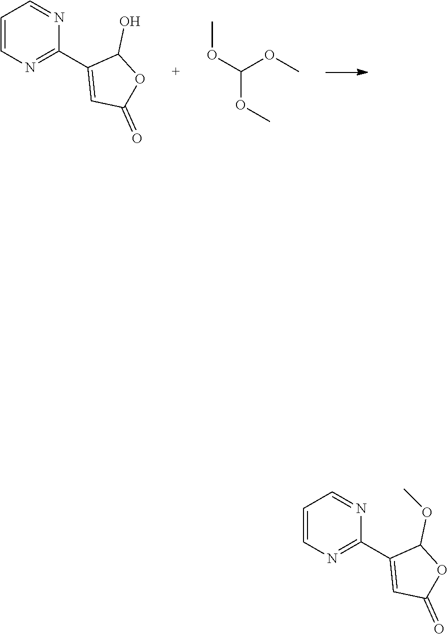

A vial was charged with 2-hydroxy-3-pyrimidin-2-yl-2H-furan-5-one (0.45 mmol, 80 mg, 1.0 eq), trimethoxymethane (0.25 mL, 2.25 mmol, 5.0 eq), p-MsOH (8.0 mg, 0.046 mmol, 0.1 eq.) and methanol (0.3 ml). The reaction was heated to 80° C. and stirred for 4 h and then stored at −20° C. overnight. Solvent was removed then the crude was purified. The crude was then purified by column chromatography (DCM/MeOH) followed by a second purification on Reverse phase chromatography (C18) to yield a white solid (12.9 mg).

¹H NMR (400 MHz, CDCl3) δ ppm 3.68 (s, 3H) 6.41 (d, J=0.73 Hz, 1H) 7.00 (d, J=1.10 Hz, 1H) 7.35 (t, J=4.77 Hz, 1H) 8.88 (d, J=5.14 Hz, 2H)

### Example 9: Preparation of 2-ethoxy-3-pyrimidin-2-yl-2H-furan-5-one 3 from 2-hydroxy-3-pyrimidin-2-yl-2H-furan-5-one

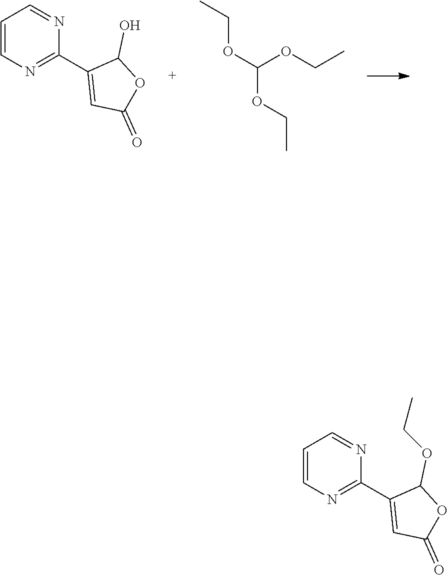

A vial was charged with 2-hydroxy-3-pyrimidin-2-yl-2H-furan-5-one (80 mg, 0.45 mmol, 1.0 eq), diethoxymethoxyethane (2.25 mmol, 0.37 mL, 5.0 eq), catalytic amount of p-MsOH (8.0 mg, 0.046 mmol, 0.1 eq.) and EtOH (0.22 mL). The reaction was heated to 80° C. and stirred for 4 h and then stored at −20° C. overnight. The solvent was then evaporated yielding a brown solid (157.4 mg). The crude was then purified by automated column chromatography (DCM/MeOH) yielding a white solid (35.6 mg). A second purification Reverse phase chromatography (C₁₈) to yield a white solid (18.1 mg).

¹H NMR (400 MHz, CDCl3) δ ppm 1.30 (t, J=6.97 Hz, 3H) 3.89-4.04 (m, 2H) 6.47 (d, J=0.73 Hz, 1H) 6.98 (d, J=0.73 Hz, 1H) 7.34 (t, J=4.95 Hz, 1H) 8.86 (d, J=4.77 Hz, 2H)

### Example 10: Preparation of 2-chloro-3-pyrimidin-2-yl-2H-furan-5-one furanone from 2-hydroxy-3-pyrimidin-2-yl-2H-furan-5-one

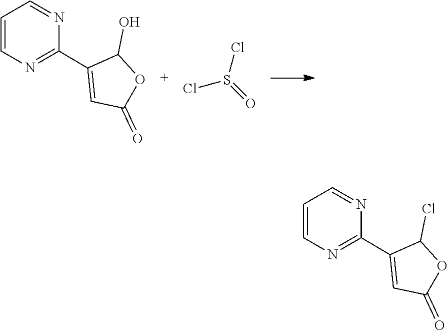

A vial was charged with 2-hydroxy-3-pyrimidin-2-yl-2H-furan-5-one (0.45 mmol, 80 mg, 1.0 eq) and dissolved with 1,2-dichloroethane (0.9 mL, 2 mL/mmol). Thionyl chloride (0.075 g, 0.63 mmol, 0.046 mL, 1.4 eq) was added dropwise and catalytic amount of DMF to the solution, which was then stirred for 1 h at 80° C. The solvent was then evaporated yielding a black solid (136.2 mg). The crude was then absorbed on isolute and purified by automated column chromatography using Cyclohexane/EtOAc to yielding a dark brown solid (35.7 mg)

¹H NMR (400 MHz, CDCl3) δ ppm 7.07 (d, J=0.73 Hz, 1H) 7.17 (d, J=1.10 Hz, 1H) 7.40 (t, J=4.77 Hz, 1H) 8.91 (d, J=5.14 Hz, 2H)

### Example 11: Preparation of 3-(6-oxo-4-pyrimidin-2-yl-pyridazin-1-yl)propanenitrile

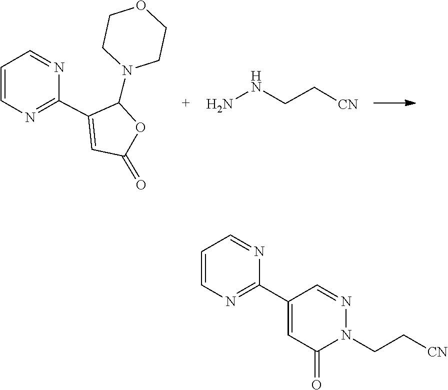 [SMILES: O=C1C=C(c2ncccn2)C(N2CCOCC2)O1 ; N#CCCNN ; N#CCCn1ncc(-c2ncccn2)cc1=O]

### General Procedure 1:

To a suspension of 2-morpholino-3-pyrimidin-2-yl-2H-furan-5-one (pure at 93% w/w) (0.25 g, 0.94 mmol) in methanol (1 ml) was added acetic acid (0.54 ml) and 3-hydrazinopropanenitrile (1.04 mmol). The yellow mixture was heated at 40° C. for 1 hour, then it was allowed to cool down to room temperature.

### Work Up:

Reaction mixture was extracted with water and ethyl acetate, the organic phase was washed once with brine, dried over sodium sulfate, filtered and evaporated under reduced pressure. 0.19 g of the title compound (purity 88% w/w as measured by Quantitative NMR) was obtained (78% chemical yield). The crude product was purified by chromatography column (DCM/MeOH gradient). 0.13 g of the title compound was isolated as a beige solid, with a purity of 99% w/w as measured by NMR.

¹H NMR (400 MHz, CDCl₃) δ ppm 2.98 (t, J=6.97 Hz, 2H) 4.53 (t, J=6.97 Hz, 2H) 7.39 (t, J=4.95 Hz, 1H) 7.97 (d, J=1.83 Hz, 1H) 8.84 (d, J=1.83 Hz, 1H) 8.90 (d, J=4.77 Hz, 2H)

### Example 12: Preparation of tert-butyl 3-(6-oxo-4-pyrimidin-2-yl-pyridazin-1-yl)propanoate

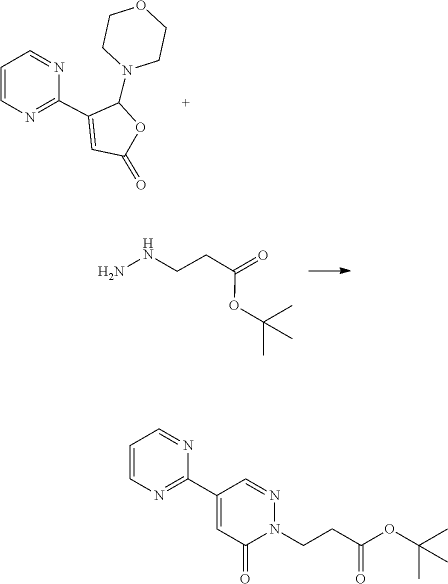 [SMILES: O=C1C=C(c2ncccn2)C(N2CCOCC2)O1 ; CC(C)(C)OC(=O)CCNN ; CC(C)(C)OC(=O)CCn1ncc(-c2ncccn2)cc1=O]

### Procedure:

The title compound was prepared according to General Procedure 1 (above), from 2-morpholino-3-pyrimidin-2-yl-2H-furan-5-one (250 mg, 1.00 mmol), tert-butyl 3-hydrazinopropanoate (185 mg, 1.09 mmol, 1.1 eq.), acetic acid (0.567 ml) in MeOH (0.932 mL). The title compound was isolated as a white solid (56 mg) after filtration, with a purity of 97.9% w/w as measured by quantitative NMR (18.3% Isolated Yield).

1H NMR (400 MHz, CDCl₃) δ ppm 1.46 (s, 9H) 2.82 (t, J=7.15 Hz, 2H) 4.50 (t, J=7.15 Hz, 2H) 7.37 (t, J=4.95 Hz, 1H) 7.93 (d, J=2.20 Hz, 1H) 8.76 (d, J=2.20 Hz, 1H) 8.89 (d, J=4.77 Hz, 2H)

### Example 13: Preparation of 3-(6-oxo-4-pyrimidin-2-yl-pyridazin-1-yl)propanoic acid

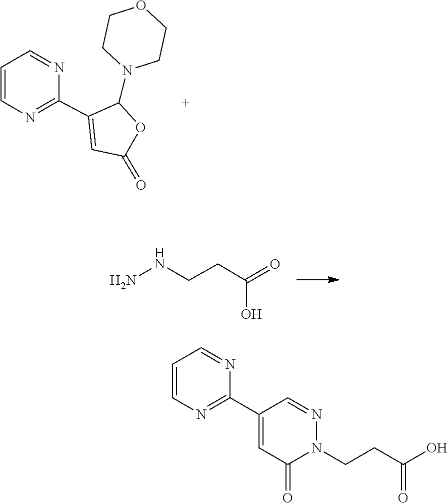 [SMILES: O=C1C=C(c2ncccn2)C(N2CCOCC2)O1 ; NNCCC(=O)O ; O=C(O)CCn1ncc(-c2ncccn2)cc1=O]

### Procedure:

The title compound was prepared according to General Procedure 1 (above), from 2-morpholino-3-pyrimidin-2-yl-2H-furan-5-one (150 mg, 0.528 mmol), 3-hydrazinopropanoic acid (91 mg, 0.581 mmol, 1.1 eq.), acetic acid (0.302 ml) in MeOH (2.1 mL). The title compound was isolated as a white solid (89 mg) after filtration. Crude recovery mass 64%.

NMR data: ¹H NMR (400 MHz, D6-DMSO) δ ppm 2.75 (t, J=7.34 Hz, 2H) 4.32 (t, J=7.15 Hz, 2H) 7.66 (t, J=4.95 Hz, 1H) 7.66 (d, J=2.20 Hz, 1H) 8.69 (d, J=2.20 Hz, 1H) 9.03 (d, J=4.77 Hz, 2H) 11.9 (bs, 1H)

### Example 14: Preparation of 2-(dimethylamino)-3-pyridazin-3-yl-2H-furan-5-one

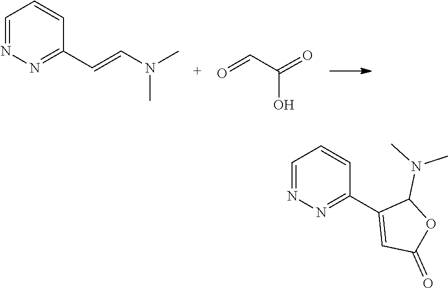

### Procedure:

To a solution of N,N-dimethyl-2-pyridazin-3-yl-ethenamine (0.300 mg, 2.0 mmol) in methanol (2 ml) was added glyoxylic acid (0.25 mL, 2.3 mmol, 1.1 eq). The mixture was heated at 50° C. overnight, then it was allowed to cool down to room temperature and then the reaction mixture filtered and concentrated in vacuo to give a black solid (0.4 g)

### Work Up:

The residue was partitioned between saturated solution of NaHCO₃ and EtOAc. The combined organic phases were dried over Na2SO4 and concentrated in vacuo to give an orange film (10.1 mg).

NMR data: 1H NMR (400 MHz, CHLOROFORM-d)) δ ppm 9.26 (dd, 1H), 7.95 (dd, 1H), 7.59 (dd, 1H), 7.13 (d, 1H), 6.18 (d, 1H), 2.46 (s, 6H)

### Example 15: Preparation of tert-butyl 3-(6-oxo-4-pyridazin-3-yl-pyridazin-1-yl)propanoate

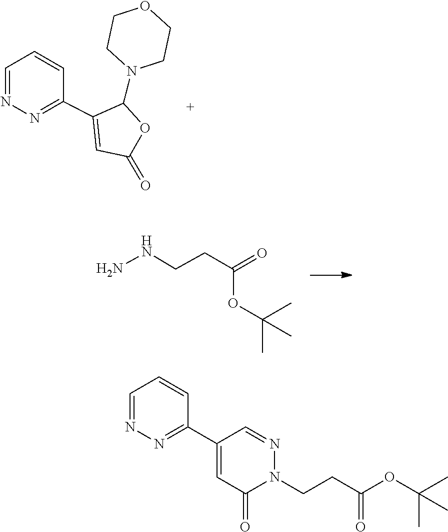 [SMILES: O=C1C=C(c2cccnn2)C(N2CCOCC2)O1 ; CC(C)(C)OC(=O)CCNN ; CC(C)(C)OC(=O)CCn1ncc(-c2cccnn2)cc1=O]

### Procedure:

To a solution of 2-morpholino-3-pyridazin-3-yl-2H-furan-5-one (0.350 mg, 1.36 mmol) in methanol (2 ml) was added 2,2,2-trifluoroacetic acid (0.11 ml, 1.36 mmol, 1 eq.) at 0° C. and then tert-butyl 3-hydrazinopropanoate (255 mg, 1.5 mmol, 1.1 eq.). The reaction mixture was allowed to warm to room temperature and left under stirring for the night.

### Work Up:

The reaction mixture was partitioned between saturated solution of water and EtOAc. The combined organic phases were dried over Na2SO4 and concentrated in vacuo to give the crude title compound with a purity of 20% w/w as measured by Quantitative NMR (18% yield).

NMR data: 1H NMR (400 MHz, CDCl3-d3) δ ppm 1.49 (s, 9H), 2.77 (t, 2H) 4.5 (t, 2H) 7.40 (s, 1H) 7.67 (dd, 1H), 7.9 (d, 1H), 8.71 (d, 1H) 9.35 (dd, 1H)

### Example 16: Preparation of 3-(6-oxo-4-pyridazin-3-yl-pyridazin-1-yl)propanenitrile

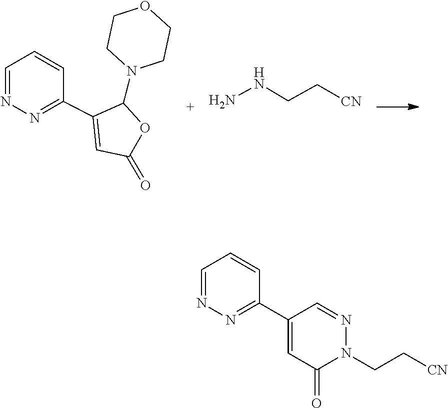 [SMILES: O=C1C=C(c2cccnn2)C(N2CCOCC2)O1 ; N#CCCNN ; N#CCCn1ncc(-c2cccnn2)cc1=O]

### Procedure:

To a solution of 2-morpholino-3-pyridazin-3-yl-2H-furan-5-one (0.100 mg, 0396 mmol) in methanol (0.375 ml) was added acetic acid (0.05 ml, 0.796 mmol, 2 eq.) and then 3-hydrazinopropanenitrile (255 mg, 1.5 mmol, 1.1 eq.). The reaction mixture was heated to 40° C. and stirred for 2 h.

### Work Up:

The reaction mixture was concentrated afford the crude title compound as a black gum with a purity of 32% w/w as measured by Quantitative NMR (60% yield).

NMR data: NMR data: 1H NMR (400 MHz, CDCl3-d3) δ ppm 3.00 (t, 2H) 4.5 (t, 2H) 7.45 (s, 1H) 7.70 (dd. 1H). 7.90 (d. 1H). 8.80 (d. 1H) 9.35 (dd. 1H)

### Example 17: Preparation of 2,2-dimethylpropyl 2-(6-oxo-4-pyridazin-3-yl-pyridazin-1-yl)ethanesulfonate

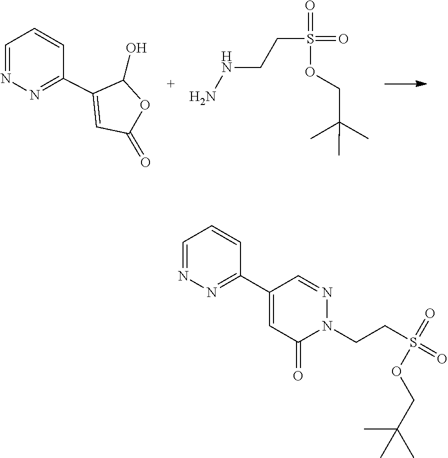 [SMILES: O=C1C=C(c2cccnn2)C(O)O1 ; CC(C)(C)COS(=O)(=O)CCNN ; CC(C)(C)COS(=O)(=O)CCn1ncc(-c2cccnn2)cc1=O]

### Procedure:

To a solution of 2-hydroxy-3-pyridazin-3-yl-2H-furan-5-one prepared freshly by mixing N,N-dimethyl-2-pyridazin-3-yl-ethenamine (120 mg, 0.67 mmol), 2-hydroxy-2-morpholino-acetic acid (175 mg, 0.945 mmol, 1.4 eq.) and acetic acid (2.4 ml) was added in one portion 2,2-dimethylpropyl 2-hydrazinoethanesulfonate (183 mg, 0.57 mmol, 1 eq.). The reaction mixture was stirred at rt for 1 h.

### Work Up:

Reaction mass was dissolved in methylene chloride then washed with saturated solution of NaHCO₃. Organics were combined and dried over MgSO4 then filtered. Organics was concentrated to dryness to afford the title compound as a black gum with a purity of 43% w/w as measured by Quantitative NMR (40% yield).

NMR data: 1H NMR (400 MHz, CHLOROFORM-d) δ ppm 9.32 (dd, J=4.77, 1.47 Hz, 1H), 8.79 (d, J=2.20 Hz, 1H), 7.90 (dd, J=8.62, 1.65 Hz, 1H), 7.65-7.72 (m, 1H), 7.42 (d, J=2.20 Hz, 1H), 4.68-4.74 (m, 2H), 3.95 (s, 2H), 3.68-3.73 (m, 2H), 1.01 (s, 9H)

### Example 18: Preparation of 4-pyrimidin-2-yl-1H-pyridazin-6-one

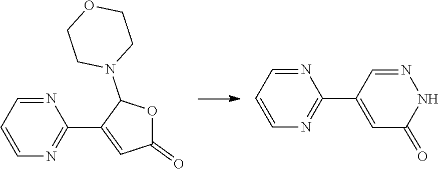 [SMILES: O=C1C=C(c2ncccn2)C(N2CCOCC2)O1 ; O=c1cc(-c2ncccn2)cn[nH]1]

To a solution of 2-morpholino-3-pyrimidin-2-yl-2H-furan-5-one (5.00 g, 19.6 mmol, 1.0 eq.) in MeOH (40 g) was added acetic acid (11.8 g, 196 mmol, 10.0 eq) at 25° C. The resulting suspension was stirred at room temperature and heated to 40° C. Hydrazine hydrate (1.09 g, 21.6 mmol, 1.10 eq.) was added over a period of 60 min via syringe pump. The resulting mixture was stirred at 40° C. for 2 h. The mixture was then allowed to cool to 24° C. and water (2.5 Vol) was added. Stirring was continued for another 1 h.

The resulting suspension was filtered through buchner funnel and the collected solid was washed with 1 vol of MeOH:Water (3.2:1). The collected solid was dried under reduced pressure at 60° C. The title compound was obtained as a solid (2.6 g, 74% yield, 97% purity as determined by quant 1 HNMR using 1,3,5 trimethoxybenzene as an internal standard)

1H NMR (400 MHz, DMSO-d6) δ ppm 7.59-7.67 (m, 2H) 8.65 (d, J=1.96 Hz, 1H) 9.02 (d, J=4.89 Hz, 2H) 13.34 (br s, 1H)

### Example 19: Preparation of ethyl 3-(6-oxo-4-pyrimidin-2-yl-pyridazin-1-yl)propanoate

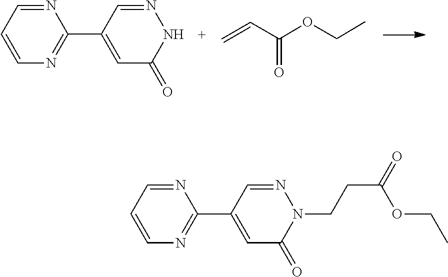

To a solution of 4-pyrimidin-2-yl-1H-pyridazin-6-one (5.00 g, 27.8 mmol, 1 eq.) in MeTHF (20 g) at room temperature was added K2CO3 (0.78 g, 5.57 mmol, 0.20 eq.) at once followed by Tetrabutylammonium bromide (0.46 g, 1.39 mmol, 0.05 eq), The reaction mixture was heated at 76° C. Ethyl prop-2-enoate (1.10 equiv., 30.6 mmol) was added dropwise via syringe pump over a period of 60 min. After end dosing, heating was continued for 10 mins and water (5 vol) was added over period of 20 min. The reaction mixture was allowed to cool to 25° C. and then cooled to 0-3° C. The resulting suspension was filtered on a sintered funnel and washed with cold (0-5° C.) water (7 vol). The solid was dried on the filter under vacuum (P=150-250 mbar) for 1 h and then under high vacuum (P=5-10 mbar at Toven=60° C.) to give the title compound (6.35 g, 98% yield, 82% purity as determined by quant 1H NMR) as a white solid.

1H NMR (400 MHz, DMSO-d6) δ ppm 1.15 (t, J=7.15 Hz, 3H) 2.81 (t, J=6.97 Hz, 2H) 4.05 (q, J=7.13 Hz, 2H) 4.35 (t, J=6.97 Hz, 2H) 7.63-7.67 (m, 2H) 8.67 (d, J=2.08 Hz, 1H) 9.02 (d, J=4.89 Hz, 2H)

### Example 20: Preparation of 4-pyridazin-3-yl-1H-pyridazin-6-one

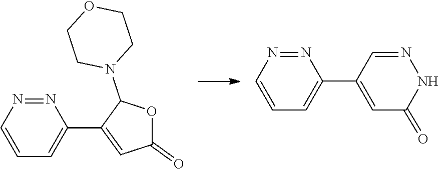 [SMILES: O=C1C=C(c2cccnn2)C(N2CCOCC2)O1 ; O=c1cc(-c2cccnn2)cn[nH]1]

To a solution of 2-morpholino-3-pyridazin-3-yl-2H-furan-5-one (5.00 g, 18.6 mmol, 1.0 eq.) in NMP (26.3 g) was added acetic acid (11.2 g, 186 mmol, 10.0 eq) at 24° C. The resulting suspension was stirred at 24° C. and then heated to 50° C. Hydrazine hydrate (1.03 g, 20.5 mmol, 1.1 eq.) was added over a period of 120 min via syringe pump at 50° C. After end of addition, the mixture was maintained at 50° C. for 2 h. The mixture was then allowed to cool to 24° C. and water (2.5 Vol) was added. Stirring was continued for another 1 h. The resulting yellow solid was filtered through a buchner funnel and washed with water (2 vol). The collected yellow solid was dried under reduced pressure at 60° C. to give the title compound (2.35 g, 69% yield, 95% purity as determined by quant 1HNMR using 1,3,5 trimethoxybenzene as internal standard)

1H NMR (400 MHz, DMSO-d6) δ ppm 7.59 (d, J=1.96 Hz, 1H) 7.89 (dd, J=8.68, 5.01 Hz, 1H) 8.40 (dd, J=8.68, 1.59 Hz, 1H), 8.66 (d, J=2.08 Hz, 1H) 9.34 (dd, J=5.01, 1.47 Hz, 1H) 13.32 (br s, 1H)

### Example 21: Preparation of ethyl 3-(6-oxo-4-pyridazin-3-yl-pyridazin-1-yl)propanoate

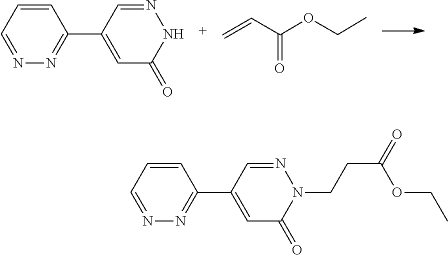

To a solution of 4-pyridazin-3-yl-1H-pyridazin-6-one (5.00 g, 27.3 mmol, 1.0 eq.) in pyridine (25 g) at 24° C. were added benzyl(triethyl)ammoniumchloride (0.32 g, 1.36 mmol, 0.05 eq.) and K2CO3 (1.52 g, 10.9 mmol, 0.40 eq.) at once. The resulting mixture was then heated at 75° C. for 1 h. Ethyl prop-2-enoate (3.03 g, 30.0 mmol, 1.1 eq.) was added dropwise via a syringe pump over a period of 4 h at 75° C. After end of addition the mixture was stirred at 75° C. for an additional hour. An additional amount of pyridine (5 mL) was then added and the mixture was then cooled to 24° C. The brown suspension was filtered through a sintered funnel to provide a brown pyridine solution of the title compound (35.2 g, 79% yield, 16.8% strength as determined by quant NMR using 1,3,5-trimethoxybenzene as an internal standard).

1H NMR (400 MHz, DMSO-d6) δ ppm 1.13-1.22 (m, 3H), 2.79-2.88 (m, 2H), 4.02-4.12 (m, 2H), 4.34-4.38 (m, 2H), 7.66-7.67 (s, 1H), 7.90-7.94 (m, 1H), 8.41-8.45 (m, 1H), 8.72 (m, 1H), 9.34-9.36 (m, 1H)

### Example 22: Preparation of 3-[2-pyrrolidin-1-ylvinyl]pyridazine from 3-methylpyridazine, triethyl orthoformate and pyrrolidine in the presence of 2,6-Di-tert-butyl-4-methylphenol as catalyst

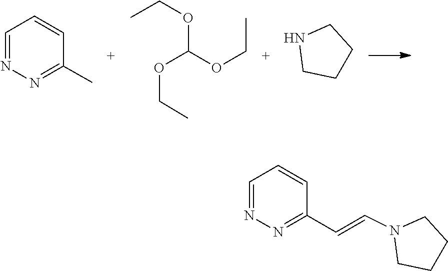

A 10 mL-microwave vial was charged with 3-methlypyridazine (0.55 g, 5.7 mmol), pyrrolidine (0.51 g, 7.2 mmol), triethyl orthoformate (1.14 g, 7.6 mmol) and 2,6-Di-tert-butyl-4-methylphenol (22 mg, 0.10 mmol, 2 mol %). The mixture was heated under stirring in a microwave reactor at 190° C. for 12 h. After cooling to room temperature, the reaction mixture was weighted, sampled and analyzed by quantitative 1H NMR (in DMSO-d6 with 1,3,5-trimethoxybenzene as standard), indicating the title compound had been formed in 55% chemical yield or 95% chemical yield based on converted starting material (58% conversion).

NMR data: 1H NMR (400 MHz, CDCl3) δ ppm: 8.60 (dd, J=4.6 Hz, 1.7 Hz, 1H), 7.80 (d, J=13.5 Hz, 1H), 7.31-7.23 (m, 2H), 5.10 (d, J=13.5 Hz, 1H), 3.28 (m, 4H), 1.88 (m, 4H).

### Example 23: Preparation of 3-[2-pyrrolidin-1-ylvinyl]pyridazine from 3-methylpyridazine, trimethyl orthoformate and pyrrolidine in the presence of 2,6-Di-tert-butyl-4-methylphenol as catalyst

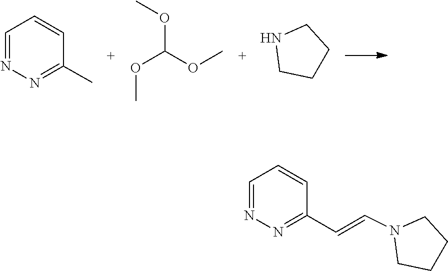

A 10 mL-microwave vial was charge with 3-methlypyridazine (0.97 g, 10 mmol), pyrrolidine (0.85 g, 12 mmol), trimethyl orthoformate (1.61 g, 15 mmol) and 2,6-Di-tert-butyl-4-methylphenol (45 mg, 0.20 mmol, 2 mol %). The mixture was heated under stirring in a microwave reactor at 200° C. for 9 h. After cooling to room temperature, the reaction mixture was weighted, sampled and analyzed by quantitative 1H NMR (in DMSO-d6 with 1,3,5-trimethoxybenzene as standard), indicating the title compound had been formed in 33% chemical yield or quantitative chemical yield based on converted starting material (33% conversion).

NMR data: 1H NMR (400 MHz, CDCl3) δ ppm: 8.60 (dd, J=4.6 Hz, 1.7 Hz, 1H), 7.80 (d, J=13.5 Hz, 1H), 7.31-7.23 (m, 2H), 5.10 (d, J=13.5 Hz, 1H), 3.28 (m, 4H), 1.88 (m, 4H).

### Example 24: Preparation of 2-[2-pyrrolidin-1-ylvinyl]pyrimidine from 2-methylpyrimidine, triethyl orthoformate and pyrrolidine in the presence of 2,6-Di-tert-butyl-4-methylphenol as catalyst

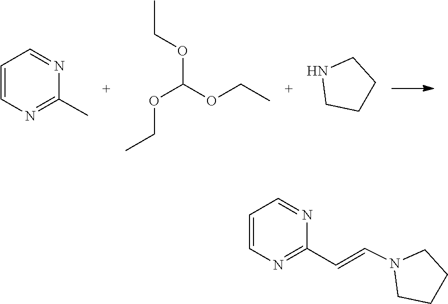

A 10 mL-microwave vial was charge with 2-methylpyrimidine (0.94 g, 10 mmol), pyrrolidine (0.85 g, 12 mmol), triethyl orthoformate (2.25 g, 15 mmol) and 2,6-Di-tert-butyl-4-methylphenol (45 mg, 0.20 mmol, 2 mol %). The mixture was heated under stirring in a microwave reactor at 220° C. for 4 h. After cooling to room temperature, the reaction mixture was weighted, sampled and analyzed by quantitative 1H NMR (in DMSO-d6 with 1,3,5-trimethoxybenzene as standard), indicating the title compound had been formed in 39% chemical yield or quantitative chemical yield based on converted starting material (39% conversion).

NMR data: 1H NMR (400 MHz, CDCl3) δ ppm: 8.34 (d, J=4.8 Hz, 2H), 7.91 (d, J=13.1 Hz, 1H), 6.75 (t, J=4.8 Hz, 1H), 5.04 (d, J=13.1 Hz, 1H), 3.28 (m, 4H), 1.88 (m, 4H).

1. A process for the preparation of a compound of formula (I): 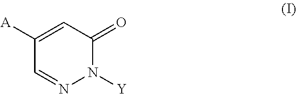
wherein
A is a 6-membered heteroaryl selected from the group consisting of formula A-I to A-VII below 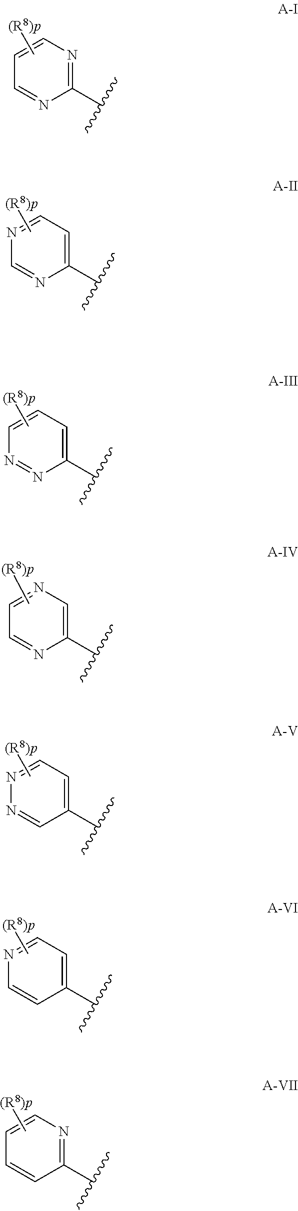
wherein the jagged line defines the point of attachment to the remaining part of a compound of formula (I), p is 0, 1 or 2; and
Y is hydrogen or the group Y-I below 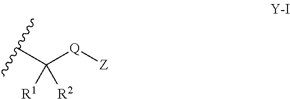
wherein the jagged line defines the point of attachment to the remaining part of a compound of formula (I); and
R¹ is hydrogen;
R² is hydrogen;
Q is (CR^{1a}R^{2b})ₘ;
m is 1;
each R^{1a} and R^{2b} are hydrogen;
Z is selected from the group consisting of —CN, —CH₂OR³, —CH(OR⁴)(OR^{4a}), —C(OR⁴)(OR^{4a})(OR^{4b}), —C(O)OR¹⁰, —C(O)NR⁶R⁷ and —S(O)₂OR¹⁰; or
Z is selected from the group consisting of a group of formula Zₐ, Z_{b}, Z_{c}, Z_{d}, Zₑ and Z_{f} below 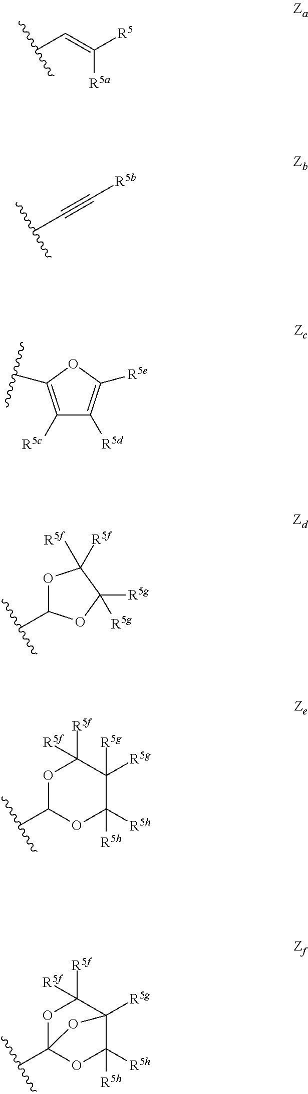
wherein the jagged line defines the point of attachment to the remaining part of a compound of formula (I); and
R³ is selected from the group consisting of hydrogen, —C(O)OR^{10a} and —C(O)R^{10a};
each R⁴, R^{4a} and R^{4b} are independently selected from C₁-C₆alkyl;
each R⁵, R^{5a}, R^{5b}, R^{5c}, R^{5d}, R^{5e}, R^{5f}, R^{5g} and R^{5h} are independently selected from the group consisting of hydrogen and C₁-C₆alkyl;
each R⁶ and R⁷ are independently selected from the group consisting of hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl; and
R^{10a} is selected from the group consisting of hydrogen, C₁-C₆alkyl, phenyl and benzyl;
said process comprising:
reacting a compound of formula (II): 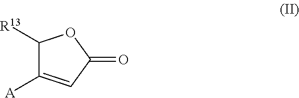
wherein A is as defined above;
R¹³ is selected from the group consisting of halogen, ═O, —OR¹⁶ and —NR¹⁴R¹⁵;
R¹⁴ and R¹⁵ are independently selected from the group consisting of hydrogen and C₁-C₆alkyl; or
R¹⁴ and R¹⁵ together with the nitrogen atom to which they are attached form a 4- to 6-membered heterocyclyl ring which optionally comprises one additional heteroatom individually selected from nitrogen, oxygen and sulfur; and
R¹⁶ is selected from the group consisting of hydrogen, C₁-C₆alkyl, —C(O)OR^{10a} and —C(O)R^{10a};
R^{10a} is as defined above;
with a compound of formula (III): 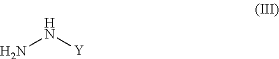
wherein Y is as defined above, to produce a compound of formula (I); 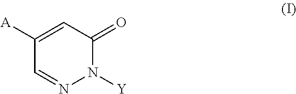
wherein A and Y are as defined above.

2. (canceled)

3. (canceled)

4. A process according to claim 1, wherein p is 0.

5. A process according to claim 1, wherein A is selected from the group consisting of formula A-Ia to A-IIIa below, 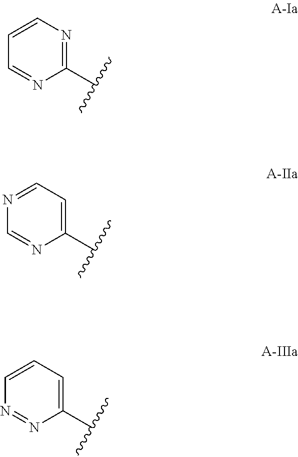 [SMILES: *c1ncccn1 ; *c1ccncn1 ; *c1cccnn1]
wherein the jagged line defines the point of attachment to the remaining part of a compound of formula (I).

6. A process according to claim 1, wherein Z is selected from the group consisting of —CN, —CH₂OH, —C(O)OR¹⁰, —S(O)₂OR¹⁰ and —CH═CH₂.

7. A process according to claim 1, wherein Z is —CN or —C(O)OR¹⁰.

8. A process according to claim 1, wherein Y is hydrogen.

9. A process according to claim 1 wherein R¹³ is selected from the group consisting of chloro, —OH, —OMe, —OEt, —N(Me)₂, pyrrolidinyl, piperidyl and morpholinyl.

10. A process according to n claim 1 wherein the compound of formula (II) is produced by:
reacting a compound of formula (IV) 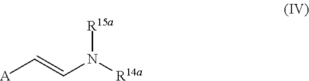
wherein A is as defined in claim 1;
R^{14a} and R^{15a} are independently selected from the group consisting of hydrogen, C₁-C₆alkyl, C₁-C₆haloalkyl and phenyl; or
R^{14a} and R^{15a} together with the nitrogen atom to which they are attached form a 4- to 6-membered heterocyclyl ring which optionally comprises one additional heteroatom individually selected from nitrogen, oxygen and sulfur;
with a compound of formula (V) 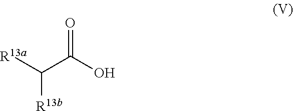
wherein each R^{13a} and R^{13b} are independently selected from the group consisting of halogen, —OR¹⁶ and —NR¹⁴R¹⁵; or R^{13a} and R^{13b} together are ═O;
and wherein R¹⁴, R¹⁵ and R¹⁶ are as defined in claim 1, to produce a compound of formula 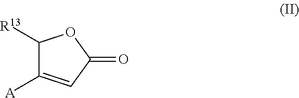
wherein A is as defined above and R¹³ is as defined in claim 1.

11. A process according to claim 10 wherein the compound of formula (IV) is produced by:
reacting a compound of formula (VI) 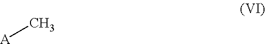
with a compound of formula (VII) 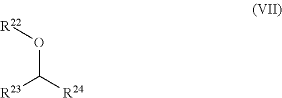
wherein R²² is C₁-C₆alkyl;
R²³ and R²⁴ are independently selected from the group consisting of C₁-C₆alkoxy and —N²⁵R²⁶;
R²⁵ and R²⁶ are independently selected from C₁-C₆alkyl; or
R²⁵ and R²⁶ together with the nitrogen atom to which they are attached form a 4- to 6-membered heterocyclyl ring which optionally comprises one additional heteroatom individually selected from nitrogen, oxygen and sulfur;
and a compound of formula (VIII) 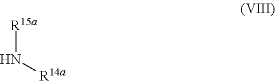
wherein R^{14a} and R^{15a} are as defined above;
to produce a compound of formula (IV) 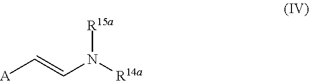
wherein A, R^{14a} and R^{15a} are as defined above.

12. A process according to claim 1 wherein the compound of formula (I) is further converted to give an agronomically acceptable salt of formula (Ia) or a zwitterion of formula (Ib), 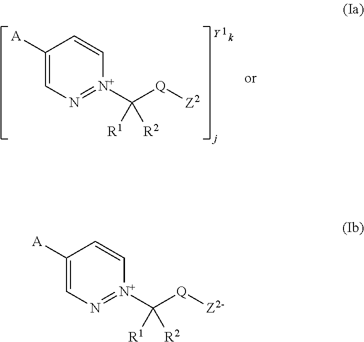
wherein Y¹ represents an agronomically acceptable anion and j and k represent integers that may be selected from 1, 2 or 3, and A, R¹, R² and Q are as defined in claim 1 and Z² is —C(O)OH or —S(O)₂OH.

13. A compound of formula (II) 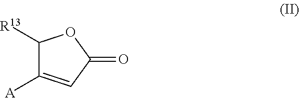
wherein A and R¹³ are as defined in claim 1.

14. Use of a compound of formula (IV) for preparing a compound of formula (I) 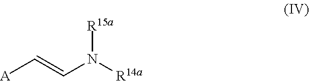
wherein A, R^{14a} and R^{15a} are as defined in claim 1.

15. A compound of formula (IV) 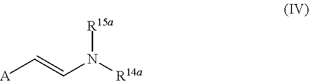
wherein A is a 6-membered heteroaryl selected from the group consisting of formula A-I, A-II, A-III, A-IV, A-V and A-VII below 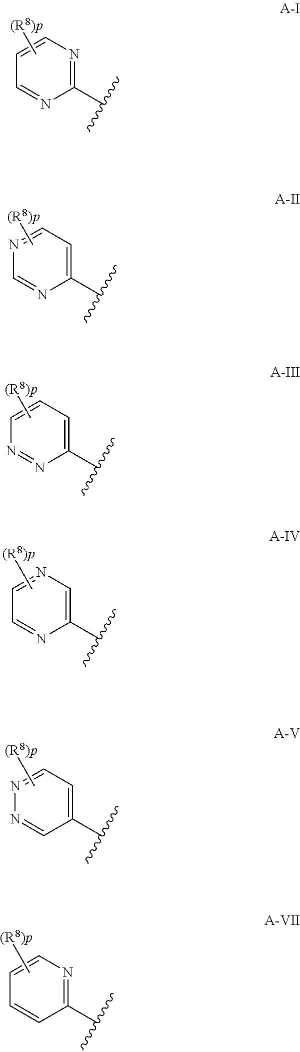
wherein the jagged line defines the point of attachment to the remaining part of a compound of formula (I), p and R⁸ are as defined in claim 1,
R^{14a} and R^{15a} are independently selected from the group consisting of C₂-C₆alkyl, C₁-C₆haloalkyl and phenyl; or
R^{14a} and R^{15a} together with the nitrogen atom to which they are attached form a 4- to 6-membered heterocyclyl ring which optionally comprises one additional heteroatom individually selected from nitrogen, oxygen and sulfur.

16. (canceled)

17. Use of a compound of formula (VI) for preparing a compound of formula (I) 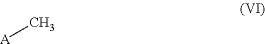
wherein A is as defined in a claim 1.

18. Use of a compound of formula (III) for preparing a compound of formula (I) 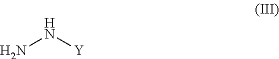
wherein Y is as defined in claim 1.
